(19) United States
(12) Patent Application Publication
(10) Pub. No.: US 2018/0346958 A1
(43) Pub. Date: Dec. 6, 2018

(54) Phage-Mediated Molecular Detection Methods and Related Aspects

(71) Applicants: Biswajit Biswas, Germantown, MD (US); Francisco Jesus Malagon Bautista, Germantown, MD (US); Luis Angel Estrella, Silver Spring, MD (US)

(72) Inventors: Biswajit Biswas, Germantown, MD (US); Francisco Jesus Malagon Bautista, Germantown, MD (US); Luis Angel Estrella, Silver Spring, MD (US)

(73) Assignee: United States of America as represented by the Secretary of the Navy, Silver Spring, MD (US)

(21) Appl. No.: 15/994,855

(22) Filed: May 31, 2018

Related U.S. Application Data
(60) Provisional application No. 62/514,534, filed on Jun. 2, 2017.

(51) Int. Cl. C12Q 1/14; C12N 7/00; C07K 14/005; C12Q 1/6806; C12Q 1/18

(52) U.S. Cl. CPC C12Q 1/14; C12N 7/00; C12Q 1/18; C12Q 1/6806; C07K 14/005

(57) ABSTRACT

Methods of detecting actively growing host organisms, including bacterial organisms, involving the detection of ribonucleic acid (RNA) products produced by unmodified infectious bioagents that infect the host organisms are provided. Related methods of assessing drug susceptibility and resistance are further provided. In addition to these surrogate detection methods, related reaction mixtures, kits, systems, and microfluidic cards are also provided.

### CROSS-REFERENCE TO RELATED APPLICATIONS

The present application claims the benefit of U.S. Provisional Patent Application No. 62/514,534 filed Jun. 2, 2017, the entire disclosure of which is incorporated by reference herein.

### STATEMENT REGARDING FEDERALLY SPONSORED RESEARCH OR DEVELOPMENT

This invention was made with government support under grant HU0001-15-2-0041 awarded by the Uniformed Services University. The government has certain rights in the invention.

### SEQUENCE LISTING

The instant application contains a Sequence Listing which has been submitted in ASCII format via EFS-Web and is hereby incorporated by reference in its entirety. Said ASCII copy, created on May 23, 2018 is named “NC105744_Sequence_List_ST25.txt” and is 2.59 kilobytes in size.

### FIELD OF THE INVENTION

The invention relates generally to molecular detection and more specifically to bacteriophage-mediated molecular detection in certain embodiments.

### BACKGROUND OF THE INVENTION

Bacteria encompass a large domain of prokaryotic microorganisms. Various species of bacteria are pathogenic and cause infectious diseases, including cholera, syphilis, tuberculosis, sepsis, and bubonic plague, among many others. Staphylococcus aureus, for example, is a gram positive (Gram+) bacteria that causes human infections. The use of antibiotics to treat S. aureus infections was initially very effective against these infections, but evolution and selection have led to the enrichment of antibiotic resistant S. aureus populations, including those resistant to methicillin and vancomycin. As an additional example, Bacillus anthracis is a spore-forming gram+ bacteria causative of anthrax, a zoonotic disease principally affecting ruminants. B. anthracis vegetative cells can differentiate under high oxygen concentrations into dormant forms that are extremely resistant to a number of environmental stresses. B. anthracis spores can survive for long periods of time and germinate once the right environmental conditions, typically involving ingestion by animals, are met. In humans, the most common natural infection transmission route is through the handling or consumption of contaminated livestock. Depending on the form of infection, anthrax in humans is classified as cutaneous (characterized by black skin ulcers), respiratory and gastrointestinal and, if untreated, the mortality rates of these forms of infection are high.

Molecular detection of bacterial infections or detection of contamination in biological, environmental, food, and other sample types, for example, by PCR using primers that target bacterial genomes, is well-known. This technique, while sensitive, is prone to generate false positives due to the presence of trace DNA in laboratory testing settings, and is generally not well suited for antibiotic susceptibility determinations. These are some of the reasons clinical testing is frequently still carried out using more time-consuming physiological assays, such as cell culture. Other exemplary molecular methods to detect bacterial infections include immunoassays that involve the use of antibodies that target specific bacterial species. These assays are relatively simple, but are typically less sensitive than PCR-based approaches.

Bacteriophages, or simply “phages”, refer to a group of extremely diverse and abundant viruses that infect bacteria. Phage infection is generally highly efficient and host specific, a fact used by researchers in strategies to deliver exogenous nucleic acids into bacteria. In the case of lytic DNA phages, for example, the injected genomic DNA molecule directs the very rapid production of a high number of progeny phages per infected cell, typically dubbed as the burst size. This is the basis for phage typing bacterial diagnostic plaque assays, further refined using labeled phages, anti-phage antibodies, or genetically engineered reporter-containing phages. The dependence of phage life cycle progression on the physiological state of the bacteria also allows for the evaluation of antibiotic resistance using, for example, phage immunodetection methods. Nevertheless, these types of antibiotic susceptibility assays require the production of suitable antibodies.

Some molecular techniques for bacterial detection involve the use of genetically engineered phages. These assays typically use phages to deliver heterologous nucleic acid cassettes to bacterial targets that function as markers for detection. These approaches also frequently rely on phage particle multiplication in the infected bacterial host cells in order to generate detectable signals. Nonetheless, the introduction of heterologous markers in phage vectors depends on the idiosyncrasy of each phage genome. In the case of phages not previously characterized for this purpose, this can be a cumbersome and slow process, particularly due to the enormous number of specific phage/bacterial host pairings.

In view of the foregoing, it is apparent that improved approaches to detect target organisms, such as bacteria, are needed.

### SUMMARY OF THE INVENTION

Bacterial infections pose a challenge to human health and burden the health care system, even more so with the spread of antibiotic-resistant populations. To provide effective treatment and improved prognosis, effective diagnostic methods are of great importance. The present invention provides Phage-Mediated Molecular Detection (PMMD) methods for the detection of bacterial organisms and for the assessment of bacterial antibiotic resistance. In certain embodiments, these techniques include a short incubation (e.g., approximately ten minutes or less in certain embodiments) of biological samples with a naturally occurring or unmodified bacteriophage (phage) targeting the bacteria of interest. In some of these embodiments, this incubation is followed by total RNA extraction and reverse transcription (RT)-PCR. As described in the examples provided herein, PMMD methods were applied to detect Staphylococcus aureus (S. aureus), a major causative agent of human bacterial infections. The results presented in these examples demonstrate that the PMMD methods provide the desired characteristics of bacterial diagnostics for use in clinical testing, among other applications. In addition, due to the dependence of the signal on the physiological state of the bacteria (e.g., the metabolic phase of the targeted S. aureus), the PMMD methods of the invention can readily distinguish between, for example, methicillin-sensitive (MSSA) and methicillin-resistant (MRSA) S. aureus strains. In addition to significantly reducing culture incubation time, the methods of the invention are highly specific and sensitive, and can be applied to complex biological samples without the need to isolate the targeted bacterial organism from other components of clinical samples.

As described herein, the PMMD methods and related aspects are well-suited for use in medical diagnostics of bacterial infections. First, the methods are able to readily differentiate antibiotic sensitive bacteria from antibiotic resistant bacteria. As one example, cefoxitin (a CDC and CLSI recommended drug for testing of MRSA strains) was used to evaluate antibiotic sensitive and antibiotic resistant strains of S. aureus. The PMMD methods of the invention are readily adaptable to similarly assess other drugs, either bactericide or bacteriostatic, since the active growth of the bacteria is needed for the phage metabolism and synthesis of phage RNA products, which can be detected. Compared with PCR approaches that amplify genetic resistance markers, the PMMD methods described herein can be used to test the resistance or sensitivity of a bacterial organism of interest to a number of different antibiotics using the same oligonucleotide primers and/or probes and does not require the use of different oligonucleotide primers and/or probes to differentiate between different genetic resistance markers. In addition, these methods also do not require different primer pairs for different resistance cassettes, and also avoid false positives due to the presence of silent or mutant cassettes.

Second, the limit-of-detection (LOD) achievable with the methods described herein is well-suited for clinical diagnostics applications. For example, strong signals have been obtained from as few as about 100 bacterial cells using the PMMD methods of the invention. It is also to be noted that the background is almost null for the uninfected bacteria controls in these applications. Highly purified phages (e.g., obtained via CsCl-gradient isolation procedures) are also used to achieve high sensitivity in certain embodiments.

Third, the PMMD methods of the invention are fast. In certain applications, for example, the implementation time for the methods is about three hours or about five hours, depending on whether bacterial detection only, or detection plus antibiotic sensitivity, is performed. This time frame is further decreased with process automation and adjusting the incubation periods depending on the antibiotic and culture media used. For example, one-hour incubation time in LB media were used to promote high bacterial metabolic rate. Significantly shorter incubation times can also be utilized (Krueger et al. (1930), “The kinetics of the bacterium-bacteriophage reaction,” J Gen Physiol., 14:223-54). Further, the incubation time of the bacteria sample and corresponding phage can be reduced significantly, for example, to 10 minutes or less, because typically the bacteriophage RNA is detected before the bacteriophage substantially replicates in the bacterial organism.

Fourth, the methods of the inventions are specific and readily applied to complex mixtures. The specificity level is generally determined by particular phage selected for use in a given application. In the case of phage K, for example, this virus infects the great majority of S. aureus strains and only propagates in a few isolates of very closely related Staphylococcus, such as S. hyicus, used sometimes as a surrogate strain for phage propagation for safety reasons (Estrella et al. (2016) “Characterization of novel Staphylococcus aureus lytic phage and defining their combinatorial virulence using the OmniLog system,” Bacteriophage, 6:e1219440). Moreover, co-infection with other S. aureus phage(s) can raise the number of positive S. aureus strains to nearly 100% (Xia et al. (2014), “Phages of Staphylococcus aureus and their impact on host evolution,” Infect Genet Evol., 21:593-601).

In summary, the PMMD methods of the invention for the detection and drug sensitivity assessment of bacterial organisms in samples of different origins are highly desirable. These methods have characteristics that make them well-suited for application in clinical and research diagnostics, including high sensitivity, high specificity, low cost and high speed.

In one aspect, the invention provides a method for detecting a host organism that is actively growing (e.g., in an anabolic growth phase). The method includes contacting a host organism with a selected unmodified infectious bioagent that infects the host organism and produces one or more ribonucleic acid (RNA) products (e.g., mRNAs or the like). The method also includes detecting at least one of the RNA products from the unmodified infectious bioagent, thereby detecting the actively growing host organism. The method typically includes detecting the RNA products before the unmodified infectious bioagent substantially replicates in the host organism. In certain embodiments, the method includes growing the host organism in a cell culture medium (or other appropriate conditions to induce or maintain the growth of the host organism) prior to the contacting step. In some embodiments, the method includes contacting one or more additional samples of the host organism with a suspected or chosen therapeutic agent prior to detecting the RNA products. In these embodiments, detection of the RNA products from the unmodified infectious bioagent in these samples at levels comparable to those in samples of infected host organisms that have not been contacted with the suspected or chosen therapeutic agent indicates that the host organism is resistant to the therapeutic agent. In some of these embodiments, the sample of host organism is contacted with the suspected or chosen therapeutic agent prior to contacting the sample of host organism with the unmodified infectious bioagent.

In a particular embodiment, the invention provides a method of detecting an actively growing bacterial organism. The method includes contacting a bacterial organism with an unmodified bacteriophage that infects the bacterial organism and produces one or more ribonucleic acid (RNA) products. The method also includes detecting at least one of the RNA products from the unmodified bacteriophage before the unmodified bacteriophage substantially replicates in the bacterial organism, thereby detecting the actively growing bacterial organism. In some embodiments, the method further comprises growing the bacterial organism in a culture medium prior to the contacting step. In some embodiments, the duration of the contacting step is about 10 minutes (or less) prior to detecting the RNA products from the unmodified bacteriophage in the detecting step. In certain embodiments, the method further comprises contacting one or more additional samples of the bacterial organism with a chosen antibiotic agent prior to detecting the RNA products. In these embodiments, detection of the RNA products from the unmodified bacteriophage in said samples at levels comparable to those detected in the infected bacterial organisms that have not been contacted with the chosen antibiotic agent indicates that the bacterial organism is resistant to the antibiotic agent. In contrast, when RNA products from the unmodified bacteriophage in said samples are not detected, or are detected at decreased levels relative to the infected bacterial organisms that have not been exposed to the chosen antibiotic agent, in these embodiments, this indicates that the bacterial organism is susceptible or sensitive to the chosen antibiotic agent. In some embodiments, the bacterial organism is contacted with the chosen antibiotic agent prior to contacting the bacterial organism with the unmodified bacteriophage.

These methods optionally include extracting or purifying the RNA products produced by the unmodified bacteriophage prior to the detecting step. Typically, this includes lysing the bacterial organism after the contacting step and prior to the detecting step. In some embodiments, these methods include amplifying the RNA products produced by the unmodified bacteriophage prior to the detecting step. Typically, this is achieved by reverse transcribing the RNA product to produce a cDNA molecule and amplifying the cDNA molecule. In certain of these embodiments, for example, the bacterial organism is Staphylococcus aureus, the unmodified bacteriophage is Bacteriophage K, and the RNA products produced by the unmodified bacteriophage are amplified prior to or during the detecting step. In some of these embodiments, the RNA products are amplified using one or more oligonucleotides comprising sequences selected from the group consisting of: SEQ ID NO: 1, SEQ ID NO: 2, SEQ ID NO: 3, SEQ ID NO: 4, SEQ ID NO: 5, SEQ ID NO: 6, and SEQ ID NO: 7. In some of these embodiments, the bacterial organism is Escherichia coli, the unmodified bacteriophage is Enterobacteria phage T4, and the RNA products produced by the unmodified bacteriophage are amplified prior to or during the detecting step. In certain of these embodiments, the RNA products are amplified using one or more oligonucleotides comprising sequences selected from the group consisting of: SEQ ID NO: 8 and SEQ ID NO: 9. In still other exemplary embodiments, the bacterial organism is Bacillus anthracis, the unmodified bacteriophage is Bacillus anthracis Gamma phage, and the RNA products produced by the unmodified bacteriophage are amplified prior to or during the detecting step. In some of these embodiments, the RNA products are amplified using one or more oligonucleotides comprising sequences selected from the group consisting of: SEQ ID NO: 10 and SEQ ID NO: 11.

In other aspects, the invention provides various reaction mixtures useful in actively growing host organisms. In some embodiments, for example, the invention provides a reaction mixture to detect Staphylococcus aureus that includes ribonucleic acid (RNA) products from an unmodified Bacteriophage K in which the one or more RNA products have been obtained from Staphylococcus aureus that was infected with the unmodified Bacteriophage K. This reaction mixture also includes one or more oligonucleotides comprising sequences selected from the group consisting of: SEQ ID NO: 1, SEQ ID NO: 2, SEQ ID NO: 3, SEQ ID NO: 4, SEQ ID NO: 5, SEQ ID NO: 6, and SEQ ID NO: 7. In other embodiments, the invention provides a reaction mixture to detect Escherichia coli that includes ribonucleic acid (RNA) products from an unmodified Enterobacteria phage T4 in which the one or more RNA products have been obtained from Escherichia coli that was infected with the unmodified Enterobacteria phage T4. This reaction mixture also includes one or more oligonucleotides comprising sequences selected from the group consisting of: SEQ ID NO: 8 and SEQ ID NO: 9. In some embodiments, the invention also provides a reaction mixture to detect Bacillus anthracis that includes ribonucleic acid (RNA) products from an unmodified Bacillus anthracis Gamma phage in which the one or more RNA products have been obtained from Bacillus anthracis that was infected with the unmodified Bacillus anthracis Gamma phage. This reaction mixture also includes one or more oligonucleotides comprising sequences selected from the group consisting of: SEQ ID NO: 10 and SEQ ID NO: 11. The reaction mixtures of the invention also typically include additional reagents, such as reverse transcriptases, DNA polymerases, labeled probes (e.g., Taqman probes, Molecular Beacon probes, Scorpion probes, etc.), buffers, and the like.

In still another aspect, the invention provides a kit to detect an actively growing target bacterial organism. The kit includes one or more containers that include (a) an unmodified bacteriophage that infects the target bacterial organism and produces one or more ribonucleic acid (RNA) products, and (b) one or more oligonucleotides to detect the RNA products. The kit also includes instructions to contact the target bacterial organism with the unmodified bacteriophage to produce the RNA products and to detect the RNA products using the oligonucleotides. To illustrate, in some embodiments, the target bacterial organism is Staphylococcus aureus, the unmodified bacteriophage is Bacteriophage K, and the oligonucleotides comprise sequences selected from the group consisting of: SEQ ID NO: 1, SEQ ID NO: 2, SEQ ID NO: 3, SEQ ID NO: 4, SEQ ID NO: 5, SEQ ID NO: 6, and SEQ ID NO: 7. In other exemplary embodiments, the target bacterial organism is Escherichia coli, the unmodified bacteriophage is Enterobacteria phage T4, and the oligonucleotides comprise sequences selected from the group consisting of: SEQ ID NO: 8 and SEQ ID NO: 9. In still other embodiments, the target bacterial organism is Bacillus anthracis, the unmodified bacteriophage is Bacillus anthracis Gamma phage, and the oligonucleotides comprise sequences selected from the group consisting of: SEQ ID NO: 10 and SEQ ID NO: 11. Optionally, the kits include instructions to contact the target bacterial organism with a chosen antibiotic agent prior to detecting the RNA products, wherein detection of the RNA products from the unmodified bacteriophage at levels comparable to those detected in infected bacterial organisms that have not been contacted with the chosen antibiotic agent indicates that the target bacterial organism is resistant to the antibiotic agent. In some of these embodiments, the kit also includes a container with the chosen antibiotic agent for use in testing the susceptibility or resistance status of the target bacterial organism.

In another aspect, the invention provides a system to detect an actively growing bacterial organism. The system comprises a reaction mixture processing component configured to contact a bacterial organism with an unmodified bacteriophage that infects the bacterial organism and produces one or more ribonucleic acid (RNA) products. The system also comprises a detection component configured to detect the RNA products from the unmodified bacteriophage before the unmodified bacteriophage substantially replicates in the bacterial organism to thereby detect the actively growing bacterial organism. In some embodiments, the system also comprises a reagent handling component configured to transfer one or more reagents to and/or from the reaction mixture processing component. In certain embodiments, the system also includes a sample preparation component configured to prepare one or more samples comprising the bacterial organism prior to being contacted with the unmodified bacteriophage and/or to purify the RNA products produced by the unmodified bacteriophage. Typically, the system also comprises a controller comprising logic instructions that direct the reaction mixture processing component to contact the bacterial organism with the unmodified bacteriophage, and the detection component to detect the RNA products.

The systems of the invention are optionally configured to detect various species of bacterial organism as well as determine their antibiotic resistance or susceptibility status. In some embodiments, for example, the bacterial organism is Staphylococcus aureus, the unmodified bacteriophage is Bacteriophage K, and the detection component comprises a nucleic acid amplification component that is configured to amplify the RNA products using one or more primer oligonucleotides (e.g., primer oligonucleotides comprising sequences selected from the group consisting of: SEQ ID NO: 1, SEQ ID NO: 2, SEQ ID NO: 3, SEQ ID NO: 4, SEQ ID NO: 5, SEQ ID NO: 6, and SEQ ID NO: 7). In other embodiments, the bacterial organism is Escherichia coli, the unmodified bacteriophage is Enterobacteria phage T4, and the detection component comprises a nucleic acid amplification component that is configured to amplify the RNA products using one or more primer oligonucleotides (e.g., primer oligonucleotides comprising sequences selected from the group consisting of: SEQ ID NO: 8 and SEQ ID NO: 9). In still other exemplary embodiments, the bacterial organism is Bacillus anthracis, the unmodified bacteriophage is Bacillus anthracis Gamma phage, and the detection component comprises a nucleic acid amplification component that is configured to amplify the RNA products using one or more primer oligonucleotides (e.g., primer oligonucleotides comprising sequences selected from the group consisting of: SEQ ID NO: 10 and SEQ ID NO: 11).

In another aspect, the invention provides a microfluidic card to detect actively growing bacterial organisms. The microfluidic card comprises a loading port configured to receive one or more samples comprising a bacterial organism. The microfluidic card also comprises a reaction chamber operably connected to the loading port, wherein said reaction chamber is configured to receive: (a) said one or more samples comprising the bacterial organism from the loading port, and wherein said reaction chamber comprises, or is configured to receive, an unmodified bacteriophage, or (b) said one or more samples comprising the bacterial organism, wherein the sample further comprises an unmodified bacteriophage, such that the unmodified bacteriophage infects the bacterial organism and produces one or more ribonucleic acid (RNA) products when the reaction chamber receives the sample. In some embodiments, microfluidic cards include, or are operably connectable to, components (e.g., shaking mechanisms or the like) that maintain suitable oxygen concentrations in one or more chambers of the microfluidic card (e.g., for applications using aerobic bacteria). The microfluidic card also includes a detection area in which detectable signals indicative of the RNA products are produced when the detection area comprises the RNA products from the unmodified bacteriophage, which detection area operably communicates with the reaction chamber. In some embodiments, the reaction chamber comprises the detection area. In addition, the microfluidic card also includes a conveyance mechanism operably connected at least to the reaction chamber. The conveyance mechanism is configured to convey the sample and/or other reagents to and/or from the reaction chamber.

The microfluidic cards of the invention are typically configured to detect various species of bacterial organisms. To illustrate, in certain embodiments, the bacterial organism is Staphylococcus aureus, the unmodified bacteriophage is Bacteriophage K, and the detection area comprises a nucleic acid amplification component that is configured to amplify the RNA products from the unmodified Bacteriophage K. In some of these embodiments, the nucleic acid amplification component is configured to amplify the RNA products using one or more oligonucleotides comprising sequences selected from the group consisting of: SEQ ID NO: 1, SEQ ID NO: 2, SEQ ID NO: 3, SEQ ID NO: 4, SEQ ID NO: 5, SEQ ID NO: 6, and SEQ ID NO: 7, when the nucleic acid amplification component comprises the oligonucleotides. In some embodiments, the bacterial organism is Escherichia coli, the unmodified bacteriophage is Enterobacteria phage T4, and the detection area comprises a nucleic acid amplification component that is configured to amplify the RNA products from the unmodified Enterobacteria phage T4. In certain of these embodiments, the nucleic acid amplification component is configured to amplify the RNA products using one or more oligonucleotides comprising sequences selected from the group consisting of: SEQ ID NO: 8 and SEQ ID NO: 9, when the nucleic acid amplification component comprises the oligonucleotides. In still other exemplary embodiments, the bacterial organism is Bacillus anthracis, the unmodified bacteriophage is Bacillus anthracis Gamma phage, and the detection area comprises a nucleic acid amplification component that is configured to amplify the RNA products from the unmodified Bacillus anthracis Gamma phage. In some of these embodiments, the nucleic acid amplification component is configured to amplify the RNA products using one or more oligonucleotides comprising sequences selected from the group consisting of: SEQ ID NO: 10 and SEQ ID NO: 11, when the nucleic acid amplification component comprises the oligonucleotides.

### BRIEF DESCRIPTION OF THE DRAWINGS

The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 schematically depicts a system to detect actively growing bacterial organisms according to one embodiment of the invention.

FIG. 2 schematically shows a microfluidic card and processing unit to detect actively growing bacterial organisms according to one embodiment of the invention.

FIGS. 3A-C schematically represent phage-mediated molecular detection method and the predicted results using primer oligonucleotides in the 5′ and 3′ ends of phage K gp125 ORF according to one embodiment of the invention. More specifically, FIG. 3A schematically illustrates an exemplary PMMD method. FIG. 3B shows the region and size of phage K gp125 amplified by the primers OK1-F (SEQ ID NO: 1) and OK1-R (SEQ ID NO: 2). The sizes of introns 1 and 2 are also indicated. FIG. 3C depicts expected gel electrophoresis results of PCR from phage K genomic DNA (DK), and from reverse transcribed phage K lysates (LK), S. aureus cells (SA) and S. aureus cells infected with phage K (SA+LK). The predicted bands (light grey rectangles) and their sizes are indicated, where the 2.6 kb band corresponds to the expected spliced mRNA and the 4.6 kb band corresponds to the expected product of regular PCR of genomic phage K DNA.

FIGS. 4A-D show results from a PMMD assay by two step RT-PCR according to one embodiment of the invention. In particular, FIG. 4A shows gel electrophoresis results of PCR from reverse transcribed phage K lysates (LK), S. aureus cells (SA) and S. aureus cells infected with phage K (SA+LK) for 5, 10, 20 and 50 minutes. PCR from phage K genomic DNA (DK) and a commercial size marker (M) are also shown. The approximate sizes of the major bands are indicated with arrows. FIG. 4B shows Sanger sequencing chromatograms of sequencing reactions of the 2.6 kb RT-PCR band shown in FIG. 4A using primers specific for the predicted exon-exon junctions. The exon junction (black font) and intervening introns (grey font) sequences are also shown. FIG. 4C shows gel electrophoresis of PMMD results, + lanes, using four USA300 strains (SA³⁰⁰⁻¹ to ⁻⁴, corresponding to clinical isolates SA1415.COI, SA1012.COI, SA1107.COI and SA1019.COI respectively). Controls without infection, − lanes, and with phage K lysate without cells (LK), as well as a DNA size marker (M), are also shown. FIG. 4D shows gel electrophoresis of PMMD results with different bacteria infected with phage K (SA: Staphylococcus aureus, SE: Staphylococcus epidermidis, SH: Staphylococcus haemolyticus, AB: Acinetobacter baumannii, PA: Pseudomonas aeruginosa, Mix: mix with all the bacterial species). M: DNA size marker.

FIGS. 5A-C show results from a PMMD assay with primer pairs OK2 and OK3 and fluorescent qPCR comparison according to one embodiment of the invention. More specifically, FIG. 5A shows the region and size of phage K gp125 amplified by the primers OK2-F (SEQ ID NO: 4) and OK2-R (SEQ ID NO: 5) (the size of intron 1 is also indicated); and gel electrophoresis results of PCR from reverse transcribed phage K lysates (LK), S. aureus cells (SA) and S. aureus cells infected with phage K (SA+LK). PCR from phage K genomic DNA (DK) and a commercial size marker (M) are also shown. The approximate sizes of the major bands are indicated with arrows. FIG. 5B shows the region phage K gp125 amplified by the primers OK3-F (SEQ ID NO: 6) and OK3-R (SEQ ID NO: 7) and gel electrophoresis. Details of the figure are as described with respect to FIG. 5A. FIG. 5C shows results of fluorescent qPCR of RNA samples amplified with the primer pairs OK2 and OK3. The infections were done with diluted S. aureus samples (10⁴) to avoid signal saturation. The relative fluorescent units (RFU), number of cycles and Cq average and S.D. values of three parallel biological replicates done in parallel are shown. As indicated, the control templates (lysate K, SA cells and water) did not produce signals with primer pair OK2 or OK3. For clarity, geometric figures are indicated on the graph lines and beside the Cq values.

FIGS. 6A-B show specificity and sensitivity results of PMMD by fluorescent qPCR using the OK2 primer pair and CsCl gradient purified phage K (K) according to one embodiment of the invention. In particular, FIG. 6A shows specificity results of the assay using mixed bacterial populations (Staphylococcus epidermidis, Staphylococcus haemolylicus, Acinetobacter baumannii, and Pseudomonas aeruginosa) (MIX) with (+) and without (−) S. aureus. The relative fluorescent units (RFU), number of cycles and Cq average and S.D. values of are shown. FIG. 6B shows sensitivity results of the assay of S. aureus (SA) samples diluted as indicated. The cfu of the higher dilution (10⁻⁷) correspond to ˜100 cells. For clarity, geometric figures are indicated on the graph lines and beside the Cq values.

FIGS. 7A-B show discrimination of MSSA versus MRSA according to one embodiment of the invention. More specifically, FIG. 7A shows fluorescent qPCR results of PMMD of the MSSA strain RN4220. The cultures were incubated with or without cefoxitin (fox) for 1 or 3 hours as indicated. The relative fluorescent units (RFU), number of cycles and Cq average and S.D. values of are shown. Uninfected cultures, purified phage K and water controls did not produce signal. FIG. 7B shows fluorescent qPCR results of PMMD of the MRSA strain SA1012.COI. The cultures were incubated with or without cefoxitin (fox) for 1 or 3 hours as indicated. Representation of the results are as shown in FIG. 7A except that double headed arrows were added to better identify the curves comparing conditions with and without fox after 1 hour and 3 hours. Uninfected cultures, purified phage K and water controls did not produce signal. For clarity, geometric figures are indicated on the graph lines and beside the Cq values.

FIG. 8 shows results of a PMMD assay of S. aureus using a bacteriophage K sensitive strain (RN4220=SA^{RN}) and two “resistant” strains (NSC-0637=SA⁶³⁷ and COI-3195=SA³¹⁹⁵) according to one embodiment of the invention. The relative fluorescent units (RFU), number of cycles and Cq average and S.D. values of are shown. For clarity, geometric figures are indicated on the graph lines and beside the Cq values.

FIG. 9 shows results of a PMMD assay of E. coli and antibiotic assessment (chloramphenicol (CM)) according to one embodiment of the invention. No signal was observed for E. coli (EC) without T4 phage interaction (T4), corresponding to background levels. For clarity, geometric figures are indicated on the graph lines and beside the Cq values.

FIG. 10 shows results of a PMMD assay of B. anthracis and antibiotic assessment (tetracycline (Tet)) according to one embodiment of the invention. No signal was observed for B. anthracis (BA) without y phage interaction (y), corresponding to background levels. For clarity, geometric figures are indicated on the graph lines and beside the Cq values.

### DETAILED DESCRIPTION

### Introduction

Infections, especially bacterial infections, pose a challenge to human health and burden the health care system, even more so since the spread of antibiotic-resistant pathogen populations. To provide effective treatment and improved prognosis, effective diagnostic methods are of great importance. In certain embodiments, the present invention provides Phage-Mediated Molecular Detection (PMMD) methods for the detection of bacterial organisms and the assessment of antibiotic resistance. In some embodiments, the methods include a brief incubation, of approximately ten minutes or less, of the biological sample with a naturally occurring (i.e., unmodified) bacteriophage (phage) targeting the bacteria of interest, followed by RNA extraction and/or purification and a step of amplifying the extracted and/or purified RNA. In certain embodiments, the PMMD methods of the invention use modified phage targeting the bacteria of interest. The bacterial diagnostic methods described herein are suitable for use in clinical testing as well as for other applications. Moreover, due to the dependence of the signal on the physiological state of the bacteria, the PMMD methods of the invention can differentiate the antibiotic susceptibility or resistance status of target bacterial organisms, such as distinguishing between methicillin-sensitive Staphylococcus aureus (MSSA) and methicillin-resistant Staphylococcus aureus (MRSA), among others. The PMMD methods of the invention reduce culture incubation time, are highly specific and sensitive, and can be applied to complex biological samples, as well as other samples such as environmental or food samples, without the need to isolate bacterial targets from clinical samples.

### Definitions

While the specification concludes with the claims particularly pointing out and distinctly claiming the invention, it is believed that the present invention will be better understood from the following description.

All percentages and ratios used herein are by weight of the total composition unless otherwise indicated herein. All temperatures are in degrees Celsius unless specified otherwise. All measurements made are at 25° C. and normal pressure unless otherwise designated. The present invention can “comprise” (open ended) or “consist essentially of” the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention.

All ranges recited herein include the endpoints, including those that recite a range “between” two values. Terms such as “about,” “generally,” “substantially,” “approximately” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value. Unless otherwise indicated, as used herein, “a” and “an” include the plural, such that, e.g., “a bioagent” can mean at least one bioagent, as well as a plurality of bioagents, i.e., more than one bioagent. As understood by one of skill in the art, the term “phage” can be use to refer to a singe phage or more than one phage. As used in this specification and the appended claims, the singular form “the” also includes plural referents unless the context clearly provides otherwise.

Where used herein, the term “and/or” when used in a list of two or more items means that any one of the listed characteristics can be present, or any combination of two or more of the listed characteristics can be present. For example, if a composition of the instant invention is described as containing characteristics A, B, and/or C, the composition can contain A feature alone; B alone; C alone; A and B in combination; A and C in combination; B and C in combination; or A, B, and C in combination. The entire teachings of any patents, patent applications or other publications referred to herein are incorporated by reference herein as if fully set forth herein.

Before describing the invention in detail, it is to be understood that this invention is not limited to particular methods, reaction mixtures, kits, microfluidic cards, or systems, which can vary. It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this invention pertains. In describing and claiming the invention, the following terminology, and grammatical variants thereof, will be used in accordance with the definitions set forth below.

The term “bioagent” is any organism, cell, or virus, living or dead, or a nucleic acid derived from such an organism, cell or virus. Examples of bioagents include, but are not limited, to cells (including but not limited to human clinical samples, bacterial cells and other pathogens), viruses, fungi, protists, parasites, and pathogenicity markers (including but not limited to: pathogenicity islands, antibiotic resistance genes, virulence factors, and toxin genes). Bioagents may be alive or dead or in a dormant state (for example, bacteria in late stationary phase or spores) and may be encapsulated or bioengineered.

The term “communicate” refers to the direct or indirect transfer or transmission, and/or capability of directly or indirectly transferring or transmitting, something at least from one thing to another thing. In some embodiments, for example, microfluidic cards include ports, channels, chambers, and the like through which sample and reagents can be transferred to perform various assay steps.

The phrase “detecting at least one of the RNA products” or the like, includes any method for detecting RNA in a sample, either directly or indirectly (e.g., detecting a cDNA corresponding to the RNA product). Typically, the RNA products are detected indirectly following reverse transcription of the RNA products into a cDNA molecule, followed by amplification of the cDNA molecule and detection of the amplified cDNA molecule corresponding to the RNA product of interest. For example, a reverse transcription polymerase chain reaction (RT-PCR) is a technique commonly used to detect RNA expression.

The phrase “substantially replicates,” in the context of an infectious bioagent (e.g., a bacteriophage) that infects a host organism (e.g., a bacterial organism), means that the infectious bioagent has appreciably multiplied the number of copies of its genome in the host organism following the initial infection event. For example, a lytic DNA phage that has produced a sufficient number of progeny phages per infected cell to produce lysis events (reached it “burst size”) in those host cells has “substantially replicated.” In the subject invention, RNA products produced by an unmodified infectious bioagent (e.g., an unmodified bacteriophage) following its infection of a host organism (e.g., a bacterial organism) are typically detected before the unmodified infectious bioagent has “substantially replicated” in the host organism. In certain embodiments, for example, RNA products produced by unmodified bacteriophage in host bacterial organisms are detected within about 120 minutes or 60 minutes of the initial infection event. Preferably, RNA products produced by unmodified bacteriophage in host bacterial organisms are detected within about 20 minutes (e.g., about 15 minutes, about 14 minutes, about 13 minutes, about 12 minutes, about 11 minutes, about 10 minutes, about 9 minutes, about 8 minutes, about 7 minutes, about 6 minutes, about 5 minutes, or less) of the initial infection event.

The term “unmodified” refers to a bioagent or phage that has not been genetically engineered through purposeful human intervention to introduce exogenous or foreign nucleic acids to control the expression of one or more heterologous proteins, such as surrogate marker expression or other artificially designed cassettes or the like. Unmodified phages include “natural” phages and their evolutionary variants that may include naturally occurring SNPs, deletion, insertions, and the like.

The term “actively growing” in the context of cellular organisms refers to a cellular organism that is in a growth phase in which cellular constituents and/or metabolites are being synthesized by the organism (e.g., an anabolic growth phase). For example, bacteria can be cultured in growth medium prior to phage infection to induce or maintain an appropriate growth phase. Exemplary techniques for determining whether bacterial organisms are actively growing are described in, for example, Krueger et al. (1930), The kinetics of the bacterium-bacteriophage reaction, J Gen Physiol., 14:223-54, Chibani-Chennoufi et al. (2004), Phage-host interaction: an ecological perspective, J Bacteriol., 186(12):3677-86, Russell et al. (1995), Energetics of bacterial growth: balance of anabolic and catabolic reactions, Microbiol. Mol. Biol. Rev., 59(1):48-62, and Haddix et al. (2008), Kinetic Analysis of Growth Rate, ATP, and Pigmentation Suggests an Energy-Spilling Function for the Pigment Prodigiosin of Serratia marcescens, J. Bacteriol., 7453-63. To further illustrate, when ATP levels are used to assess bacterial growth phases, various kits are commercially available to determine ATP levels from various suppliers, including Isogen Life Science B.V., Thermo Fisher Scientific Inc., and Sigma-Aldrich Co. LLC.

### Phage-Mediated Molecular Detection Methods

The Phage-Mediated Molecular Detection (PMMD) methods of the invention are generally directed at the detection of actively growing bacterial organisms in various embodiments that are mediated by unmodified bacteriophage. The PMMD methods described herein detect essentially only actively growing bacteria, because the active growth of host bacteria is typically needed for bacteriophage metabolism upon infection. Further, the use of unmodified bacteriophage has various advantages, including avoiding the additional steps of introducing exogenous nucleic acid cassettes into phage particles, as required by other detection techniques.

The PMMD methods of the instant invention typically include incubating samples suspected of including bacterial organisms of interest with unmodified bacteriophage that infect those target organisms. The turn-around time of these methods (e.g., length of time from sample-to-answer) is typically very rapid, in part, because incubation times are generally of relatively brief duration (e.g., about 10 minutes or less in certain embodiments). Longer incubation times can be used but are usually not needed to generate sufficient signal for detection. In these processes, ribonucleic acid (RNA) products (e.g., mRNA or the like) produced by the unmodified bacteriophage in infected host bacterial organisms are detected and thus, indicative of the presence of actively growing target bacterial organisms in the sample. In some embodiments, the methods of the invention include RT-PCR/qPCR strategies that specifically target spliced mRNA products from which introns have been excised as opposed to unspliced or pre-mRNA products, incorrectly spliced mRNA products, and/or genomic nucleic acids. In other embodiments, RT-PCR/qPCR strategies that specifically target pre-mRNA products or intronless RNAs may be employed. The RNA products are typically extracted from the targeted bacteria for detection before the bacteriophage had formed a progeny of novel matured particles (e.g., reached its burst size, in the case of lytic DNA phages) in the infected host bacterial organism. Once extracted, the RNA products are typically subjected to an amplification step, such as reverse transcription-PCR (RT-PCR) and detected, for example, in real time-PCR reactions (qPCR) in some embodiments.

In some embodiments, the PMMD methods are additionally used to assess the antibiotic resistance or susceptibility status of the targeted bacterial organism. In these applications, the bacterial organism is typically exposed to an antibiotic agent prior to detecting RNA products produced by the unmodified bacteriophage. If RNA products are subsequently detected at levels comparable to those detected in infected bacterial organisms that have not been contacted with the suspected/chosen antibiotic agent (i.e., in samples of the infected bacterial organisms that have not been treated with the suspected/chosen antibiotic agent), this indicates that the bacterial organism is viable and, thus, resistant to the antibiotic agent. In contrast, if RNA products are not detected, or detected at decreased levels relative to samples of the infected bacterial organism that have not been exposed to the suspected antibiotic agent, this indicates that the bacterial organism is not actively growing and, thus, susceptible or sensitive to the antibiotic agent. Additional details related to performing the methods of the invention are illustrated in the examples provided herein.

### Sample Types

Essentially any sample type can be used in performing the PMMD methods of the invention. For example, samples suspected of including target bacterial organisms may include, but are not limited to, tissue or fluid isolated from one or more subjects or individuals, in vitro cell culture constituents, as well as clinical samples. Exemplary samples include blood (e.g., direct whole blood specimens, cultured blood specimens, etc.), plasma, serum, urine, synovial fluid, seminal fluid, seminal plasma, prostatic fluid, vaginal fluid, cervical fluid, uterine fluid, cervical scrapings, amniotic fluid, anal scrapings, mucus, sputum, tissue, and the like. Environmental samples (e.g., soil, waste water, food products, pharmaceutical quality control samples, and the like) are also optionally used. In the case of B. anthracis, for example, samples are typically obtained using swabs or wipes to collect spores from a given surface. Examples of B. anthracis sample collection techniques are also described in, for example, Sanderson et al. (2002), “Surface Sampling Methods for Bacillus anthracis Spore Contamination,” Emerg Infect Dis. 8(10):1145-1151.

Additional details related to suitable sample types as well as sample collection and processing are described, for example, in Guder et al., Diagnostic Samples: From the Patient to the Laboratory: The Impact of Preanalytical Variables on the Quality of Laboratory Results, 4^{th} Ed., Wiley-Blackwell (2009) and Coonrod et al. (Editors), The Direct Detection of Microorganisms in Clinical Samples, Academic Press (2012).

### Bacteriophages and Target Bacterial Organisms

A wide variety of unmodified bacteriophages that infect an array of specific host bacterial organisms are optionally used in performing the PMMD methods of the invention. To illustrate, non-limiting examples of bacteriophages and corresponding target bacterial organisms that are specifically infected by those bacteriophages are as follows:

| | TARGET BACTERIAL |
|---|---|
| BACTERIOPHAGES | ORGANISMS |
| Enterobacteria phage λ | Escherichia coli |
| Enterobacteria phage T4 | Escherichia coli |
| Enterobacteria phage T2 | Escherichia coli |
| Bacteriophage T7 | Escherichia coli |
| Enterobacteria phage R17 | Escherichia coli |
| Bacteriophage MS2 | Escherichia coli |
| Bacteriophage f2 | Escherichia coli |
| Bacteriophage Qβ | Escherichia coli |
| Bacteriophage M13 | Escherichia coli |
| Enterobacteria phage G4 | Escherichia cali |
| P1 phage | Escherichia coli |
| Bacteriophage P2 | Escherichia coli |
| Enterobacteria phage P4 | Escherichia coli |
| Enterobacteria phage phiX174 | Escherichia coli |
| Bacteriophage N4 | Escherichia coli |
| Bacteriophage K | Staphylococcus aureus |
| Bacillus anthracis | Bacillus anthracis |
| Gamma phage | |
| Bacteriophage T12 | Streptococcus pyogenes |
| Bacillus phage phi29 | Bacillus subtilis |
| Mycobacteriophage | Mycobacterium tuberculosis |
| Yersinia pestis | Yersinia pestis |
| Bacteriophage φA1122 | |

Additional details related to bacteriophages and their preparation for analysis that are optionally used in performing the PMMD methods of the invention are described in, for example, Clokie et al. (Editors), Bacteriophages: Methods and Protocols, Volume 1: Isolation, Characterization, and Interactions (Methods in Molecular Biology), 2009 Edition, Humana Press (2009), Szybalski et al. (Editors), Bacteriophages, Part B, Volume 83 (Advances in Virus Research), 1st Edition, Academic Press (2012), Abedon, The Bacteriophages, 2nd Edition, Oxford University Press (2005), and Birge, Bacterial and Bacteriophage Genetics, 4th Edition, Springer (2000).

### Bacterial Cell Culture

In certain embodiments, bacterial cells are cultured prior to being infected with unmodified bacteriophage under conditions that induce an anabolic growth phase in the targeted bacterial organism. One exemplary set of conditions that may be used when antibiotic sensitivity is not being assessed include growing bacterial cultures for one hour in LB-Lennox (10 g/l Tripone, 5 g/l yeast extract and 5 g/l NaCl) media at 37° C. with strong aeration (225 rpm shaking). For antibiotic sensitivity assays, an exemplary set of conditions that may be used include growing the bacterial cultures for one to three hours in LB-Lennox media at 37° C. with strong aeration, followed by adding the antibiotic agent to be tested to the culture media at the desired concentration. Bacterial cell culture is further described in the examples provided herein. Additional details related to bacterial cell culture are described in, for example, Ball, Bacterial Cell Culture: Essential Data (Essential Data Series), 1st Edition, Wiley (2009).

### Pre-Infection Bacteriophage Sample Preparation

Bacteriophage are prepared for use in performing the bacterial detection methods of the invention according to various approaches well-known to those of skill in the art. In some embodiments, for example, phage lysates are utilized and prepared following standard procedures. An exemplary procedure for producing phage lysates is also described in the examples provided herein. Additional methods for producing suitable phage lysates are also described in, for example, Clokie et al. (Editors), Bacteriophages: Methods and Protocols, Volume 1: Isolation, Characterization, and Interactions (Methods in Molecular Biology), 2009 Edition, Humana Press (2009). In other exemplary embodiments, bacteriophage particles are purified prior to being used in the PMMD methods of the invention. Optionally, CsCl gradients are used for phage purification as described in, for example, Estrella et al. (2016), “Characterization of novel Staphylococcus aureus lytic phage and defining their combinatorial virulence using the OmniLog system,” Bacteriophage, 6:e1219440. The use of this purification process is further described in the examples provided herein.

### Post-Infection Sample Processing

Once phage preparations have been incubated with bacterial samples to effect infection and RNA product generation by the phage in the bacteria, the bacterial cells are typically lysed and the RNA products extracted. Bacterial cells are lysed using any of a variety of well-established techniques. Examples of such cell lysis techniques include mechanical disruption, liquid homogenization, high frequency sound waves, freeze/thaw cycles, and manual grinding, among other approaches. These processes are optionally adapted for use in performing the PMMD methods of the invention. Similarly, various techniques for extracting and otherwise preparing RNA for further analysis are well-known to those of ordinary skill in the art. In addition, systems and kits suitable for use in effecting bacterial cell lysis and/or RNA extraction are commercially available. Illustrations of suitable cell lysis and RNA extraction methods, systems, and kits are also provided in the examples provided herein.

RT-PCR and qPCR Reactions and Analysis

The PMMD methods of the invention typically also include generating cDNA from extracted phage RNA products, which cDNA is then amplified and detected, for example, using real-time PCR (qPCR) techniques. Numerous RT-PCR and qPCR protocols (including various labeled probe nucleic acid configurations (e.g., TaqMan or 5′-nuclease probes, Molecular Beacon probes, Scorpion Probes, etc.)) are well-known and readily adapted for use in performing the methods of the invention. Exemplary RT-PCR and qPCR protocols, primer oligonucleotides, polymerase enzymes, thermal cyclers, and other reagents suitable for use in performing the PMMD methods of the invention are also described in the examples provided herein. Additional details regarding RT-PCR and qPCR protocols that are adapted for use in performing the methods of the invention are also described in, for example, Kochanowski et al. (Editors), Quantitative PCR Protocols (Methods in Molecular Medicine), 1st Edition, Humana Press (1999), Shafique, Polymerase Chain Reaction: Procedure, Principles, Real time PCR, Optimization, Applications, PCR Arrays, Array System Performance, Protocol, Variations, LAP LAMBERT Academic Publishing (2012), Innis et al. (Editors), PCR Applications: Protocols for Functional Genomics, 1 st Edition, Academic Press (1999), King (Editor), RT-PCR Protocols: Second Edition (Methods in Molecular Biology), 2nd edition, Humana Press (2010), and Sachse et al. (Editors), PCR Detection of Microbial Pathogens (Methods in Molecular Biology), 1 st Edition, Humana Press (2002).

Various approaches can be utilized by one of ordinary skill in the art to design oligonucleotides (e.g., primer oligonucleotides and probe oligonucleotides) that selectively bind to target nucleic acids for amplification and detection. To illustrate, the DNAstar software package available from DNASTAR, Inc. (Madison, Wis.) can be used for sequence alignments. For example, nucleic acid sequences for target bacteriophage mRNAs can be uploaded into DNAstar EditSeq program as individual files. Pairs of sequence files can be opened in the DNAstar MegAlign sequence alignment program and the Clustal W method of alignment can be applied. The parameters used for Clustal W alignments are optionally the default settings in the software. Another example algorithm that is suitable for determining percent sequence identity is the BLAST algorithm (e.g., Primer-BLAST). Software for performing versions of BLAST analyses is publicly available through the National Center for Biotechnology Information on the world wide web at ncbi.nlm.nih.gov/ as of May 30, 2017. Many other sequence alignment algorithms and software packages are also optionally utilized. Sequence alignment algorithms are also described in, e.g., Mount, Bioinformatics: Sequence and Genome Analysis, Cold Spring Harbor Laboratory Press (2001), and Durbin et al., Biological Sequence Analysis: Probabilistic Models of Proteins and Nucleic Acids, Cambridge University Press (1998.)

Some specific exemplary primer oligonucleotides that are useful in performing the PMMD methods of the invention to detect Staphylococcus aureus, Escherichia coli, and Bacillus anthracis are as follows (note: their use is further described in the examples provided herein):

Primer Oligonucleotides for Staphylococcus aureus Detection

| | | |
|---|---|---|
| OK1-F | 5′-GAGTTGGTAGATAACATTG-3′ | SEQ ID NO: 1 |
| OK1-R | 5′-AATTGTTCATCTGTTAGTTTACCTGACTCTTTATAATC-3′ | SEQ ID NO: 2 |
| SeqI2-F | 5′-CTACAATGAGAGAGCACTGG-3′ | SEQ ID NO: 3 |
| OK2-F | 5′-GAATTGCTTAAGAATTGGTTAGCTAG-3′ | SEQ ID NO: 4 |
| OK2-R | 5′-CCTGAATACTCTCAAAATCTTTAAAG-3′ | SEQ ID NO: 5 |
| OK3-F | 5′-ACAAGAAAGCTATTGGTTATGCGTTAGATAATTTAG-3′ | SEQ ID NO: 6 |
| OK3-R | 5′-TGTAGC-ATATCAGGGTCTTTAGTAAATAATCCGA-3′ | SEQ ID NO: 7 |

Primer Oligonucleotides for Escherichia coli Detection

| | | |
|---|---|---|
| T4nrdB-f | 5′-CTTTACCTCTGTTTGCACGTT-3′ | SEQ ID NO: 8 |
| T4nrdB-rev | 5′-CATTACCTTCCATGATTTCCATGT-3′ | SEQ ID NO: 9 |

Primer Oligonucleotides for Bacillus anthracis Detection

| | | |
|---|---|---|
| Gamma | 5′-GTATACATTGCAGGGATTCAAG-3′ | SEQ ID NO: 10 |
| pol-f | | |
| Gamma | 5′-CATGTTCGTCATTTAATTTCTCACC-3 | SEQ ID NO: 11 |
| pol-rev | | |

### Antibiotic Sensitivity and Resistance Testing

In addition to detecting the presence of actively growing target bacterial organisms using the PMMD methods of the invention, the sensitivity or resistance status of those target bacterial organism is also optionally readily assessed for essentially any antibiotic agent. In these embodiments, a sample that includes the targeted bacterial organism is mixed with a suspected or chosen antibiotic agent to be tested prior to detecting the RNA products produced by the bacteriophage (e.g., prior to infecting the targeted bacterial organism with the unmodified bacteriophage). Antibiotic sensitivity is indicated when RNA products produced by the bacteriophage are not detected, or detected at decreased levels relative to samples of the infected bacterial organism that have not been exposed to the chosen antibiotic agent, following this antibiotic exposure. In contrast, antibiotic resistance is indicated, when RNA products produced by the bacteriophage are detected at levels comparable to those detected in infected bacterial organisms that have not been contacted with the chosen antibiotic agent following such exposure. As one illustration, this approach was used to differentiate between MSSA and MRSA strains of S. aureus upon exposure to cefoxitin as further described in the examples provided herein.

### Reaction Mixtures

Embodiments of the invention also include various reaction mixtures that are useful to detect targeted bacterial organisms. In general, a given reaction mixture typically includes at least the RNA products (e.g., mRNAs) produced by the unmodified bacteriophage in the infected host bacterial organism (e.g., following bacterial cell lysis and RNA extraction in some embodiments) and/or corresponding cDNAs derived from those RNA products (e.g., following one or more reverse transcription steps). These reaction mixtures also include one or more primer oligonucleotides that hybridize (under selected conditions) with the RNA products and/or the corresponding cDNAs derived from those RNA products sufficiently to function as primers in polymerase-mediated extension reactions. Some specific examples of reaction mixtures of the present invention include one or more of the primer oligonucleotides provided herein (i.e., including sequences corresponding to SEQ ID NOS: 1-11).

In some embodiments, reaction mixtures include solutions containing other reagents necessary to carry out an amplification reaction (e.g., RT-PCR, qPCR, etc.) such as a thermostable RNA dependent DNA polymerase, thermostable DNA polymerase, a labeled probe oligonucleotide, dNTP's, and a divalent metal cation in a suitable buffer. A reaction mixture is referred to as complete if it contains all reagents necessary to carry out the reaction, and incomplete if it contains only a subset of the necessary reagents. It will be understood by one of ordinary skill in the art that reaction components are routinely stored as separate solutions, each containing a subset of the total components, for reasons of convenience, storage stability, or to allow for application-dependent adjustment of the component concentrations, and, that reaction components are combined prior to the reaction to create a complete reaction mixture. Furthermore, it will be understood by one of ordinary skill in the art that reaction components are packaged separately for commercialization and that useful commercial kits may contain any subset of the reaction components.

### Kits

The invention also provides various types of kits that are useful in detecting target bacterial organisms and in certain embodiments, also in assessing the antibiotic sensitivity or resistance status of those bacterial organisms. Typically, a kit includes containers that include an unmodified bacteriophage that infects the target bacterial organism and produces one or more ribonucleic acid (RNA) products. A kit also includes one or more oligonucleotides to detect those RNA products as well as instructions for doing so. Some specific examples of kits of the present invention include one or more of the primer oligonucleotides provided herein (i.e., including sequences corresponding to SEQ ID NOS: 1-11). When antibiotic sensitivity/resistance is also to be evaluated, kits also include instructions for contacting a chosen antibiotic agent with a target bacterial organism as part of those processes and optionally, a container that includes the chosen antibiotic agent to be tested. In some embodiments, kits include microfluidic cards as described herein.

In some embodiments, kits may also include suitably packaged reagents and materials needed for DNA or RNA immobilization, hybridization, and/or detection, such solid supports, buffers, enzymes, and DNA standards, as well as additional instructions for conducting the given assay. Optionally, nucleic acid detection reagents (e.g., probe oligonucleotides) are provided already attached or otherwise immobilized on solid supports (e.g., a plate, a microwell plate, a bead, a microbead, a magnetic bead, a tube (e.g., a microtube, etc.), a fiber, a whisker, a comb, a hybridization chip, a membrane, a single crystal, a ceramic layer, a self-assembling monolayer, or the like). As another option, such nucleic acid detection reagents are provided free in solution in containers, e.g., for performing the detection methods of the invention in the solution phase. In some of these embodiments, these nucleic acid detection reagents of the kits include labels and/or quencher moieties, such as when molecular beacons, 5′-nuclease probes, or the like are to be used for detection.

In these embodiments, kits also include one or more nucleotides, e.g., for use in amplifying the target nucleic acids (e.g., bacteriophage mRNAs). Optionally, at least one of the nucleotides comprises a label. In some of these embodiments, the kits further include at least one pyrophosphatase (e.g., a thermostable pyrophosphatase), e.g., for use in minimizing pyrophosphorolysis, uracil N-glycosylase (UNG) (e.g., a thermostable UNG), e.g., for use in applications where protection against carry-over contamination is desirable. In some embodiments, kits include lytic enzymes useful in performing RNA extraction steps (e.g., lysostaphin). Lytic enzymes are typically used in certain protocols in which the RNA products are extracted from infected gram-positive bacteria, such as S. aureus. In other exemplary embodiments, kits include various controls, such as bacterial control strains (e.g., MSSA and MRSA strains for antibiotic sensitivity/resistance testing).

### Systems and Microfluidic Cards

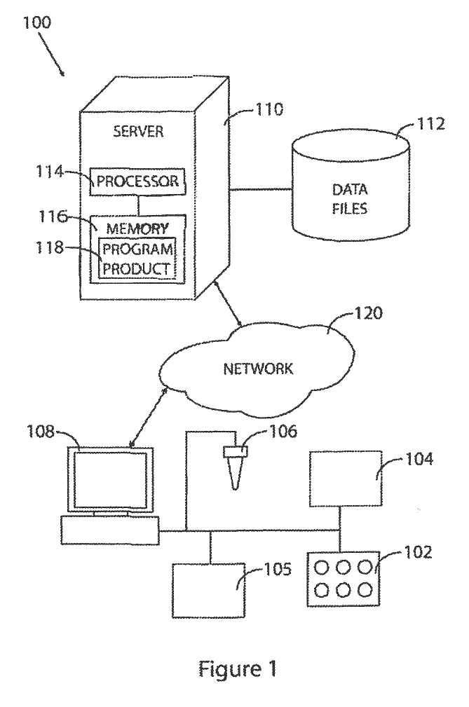

The present invention also provides various systems and related computer program products or machine readable media. In some embodiments, for example, the PMMD methods of the invention are optionally performed or facilitated at least in part using systems, cloud computing services, computer program products, machine readable media, and/or the like. To illustrate, FIG. 1 provides a schematic diagram of an exemplary system suitable for use with certain embodiments of the invention. As shown, system 100 includes reaction mixture processing component 102 schematically illustrating wells for contacting samples of target bacterial organisms with unmodified bacteriophage such that the phage infects the bacterial organisms and produces RNA products, such as mRNAs. As also shown, system 100 includes detection component 104, which is configured to detect the RNA products and thereby detect actively growing bacterial organisms after the RNA products have been extracted from the infected bacterial organisms at sample preparation component 105. System 100 also includes reagent handling component 106 (schematically shown as a robotic pipetting arm) that is configured to transfer samples and reagents to and/or from reaction mixture processing component 102, detection component 104, and sample preparation component 105. As also shown, reagent handling component 106, reaction mixture processing component 102, detection component 104, and sample preparation component 105 are all operably connected to controller 108 (shown as a computer). Controller 108 includes or accesses logic instructions that direct these system components to perform embodiments of the PMMD methods of the invention.

Detection components are structured to detect detectable signals produced, e.g., in or proximal to another component of the system (e.g., in a container, on a solid support, etc.). Suitable detection components that are optionally utilized, or adapted for use, in these systems detect, e.g., fluorescence, phosphorescence, radioactivity, absorbance, refractive index, luminescence, or the like. Detection components optionally monitor one or a plurality of signals from upstream and/or downstream of the performance of, e.g., a given assay step. For example, detection components optionally monitor a plurality of optical signals, which correspond in position to “real-time” results (e.g., from qPCR assays to detect the phage mRNAs produced using the PMMD methods of the invention). Example detection components or sensors include photomultiplier tubes, CCD arrays, optical sensors, temperature sensors, pressure sensors, pH sensors, conductivity sensors, scanning detectors, or the like. Each of these as well as other types of sensors is optionally readily incorporated into the systems described herein. Optionally, the systems of the present invention include multiple detection components.

More specific exemplary detection components that are optionally utilized in these systems include, e.g., a resonance light scattering detector, an emission spectroscope, a fluorescence spectroscope, a phosphorescence spectroscope, a luminescence spectroscope, a spectrophotometer, a photometer, and the like. Typically, thermal cycling components are integrated with, or positioned sufficiently proximal to, detection components such that qPCRs can be monitored continuously in real-time. Detection and other system components that are optionally included in the systems of the invention are described further in, e.g., Currell, Analytical Instrumentation: Performance Characteristics and Quality, John India Pvt Ltd, (2010).

In some embodiments, the systems of the invention are stand-alone in which controllers or other system components are not operably connected to external communication components. In other embodiments, controller 108 is operably connected to server 110, which includes processor 114 and memory 116, via electronic communication network 120, such as the Internet. System 100 also includes program product 118 stored on a machine readable medium, such as, for example, one or more of various types of memory that is readable by the server 110, to facilitate a guided navigational search by controller 108. Program product 118 may include logic instructions useful in analyzing data received from detection component 104 via controller 108 to identify detected bacteria organisms and to correlate that data with other systems (not shown) connected via network 120 to provide, for example, epidemiological information regarding the scope of infection in a geographic region. As shown, system 100 also includes database 112 operably connected to server 110 having data stored thereon searchable either directly or through server 110. Database 112 may have accessible data files that assist in the further characterization of detected bacterial organisms.

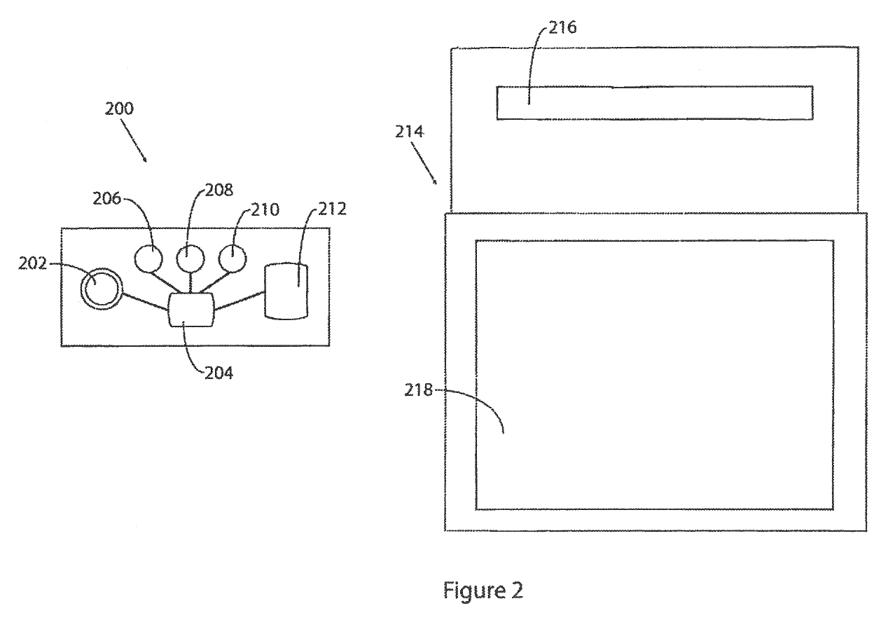

In other aspects, the invention also provides microfluidic cards and devices for performing the PMMD methods on those cards. As schematically illustrated in FIG. 2, for example, microfluidic card 200 includes loading port 202 configured to receive samples of bacterial organisms (e.g., using a swab or other sample collection device). As also shown, microfluidic card 200 also includes reaction chamber 204 operably connected (in communication with) loading port 202 via a microchannel (shown as a black line). In some embodiments, reaction chamber 204 is configured to receive the bacterial sample from loading port 202 before the sample is contacted with an unmodified bacteriophage. In these embodiments, reaction chamber 204 includes pre-loaded phage or is configured to receive the phage via a microchannel in communication with reagent storage chamber 206 or 208 to effect bacterial and unmodified phage mixing or contact in reaction chamber 204. In other embodiments, bacterial samples and bacteriophage are mixed with one another prior to being loaded into microfluidic card 200 via loading port 202 and then conveyed into reaction chamber 204. Once samples and bacteriophage have been incubated in reaction chamber 204 for a selected amount of time (e.g., about ten minutes or less in some embodiments), RNA products produced by the bacteriophage in actively growing infected bacterial targets are then extracted either in reaction chamber 204 or in a detection area 212. Although not shown in FIG. 2, in some embodiments, reaction chambers include detection areas such that all methods steps are performed in the reactions chambers. Detection area 212 typically includes a thermal cycling component (e.g., a thermoelectric cooler or the like) to effect temperature cycling for nucleic acid amplification (e.g., qPCR) and a detection component (e.g., an optical sensor) in operable proximity to detection area 212 so that detectable signals produced during the nucleic acid amplification cycles are detected in real-time. As also shown, microfluidic card 200 also includes waste chamber 210 into which waste reagents are conveyed from reaction chamber 204 at one or more steps during the performance of a given PMMD process on microfluidic card 200. Although not within view, microfluidic card 200 includes an operably connected conveyance mechanism (e.g., membrane-based valves disposed between layers of microfluidic card 200 or the like) that convey samples and other reagents to and/or from any of the ports, chambers, areas, and microchannels of microfluidic card 200.

FIG. 2 also schematically depicts a hand-held, point-of-care device for performing PMMD methods using microfluidic card 200. As shown, processing unit 214 include touch LED screen 218 for inputting instructions and other information into processing unit 214 and viewing information on LED screen 218 (e.g., the results of a give PMMD assay performed using processing unit 214. As also shown, processing unit 214 also includes microfluidic card receiving area 216, which is configured to receive microfluidic card 200 for processing bacterial samples. Although not within view, processing unit 214 includes well-known internal components that effect movement of reagents on microfluidic card 200 (e.g., pneumatic pumps and the like) and the detection of detectable signals from detection area 212. Additional details regarding microfluidic assays, cards, and associated processing units are described in, for example, Chu et al. (2017), Microfluidics for Advanced Functional Polymeric Materials, 1st Edition, Wiley-VCH, (2017), Girl (2017), Laboratory Methods in Microfluidics, 1st Edition, Elsevier, (2017), and Lu et al. (2016), Microfluidic Methods for Molecular Biology, 1st Edition, Springer, (2016).

### EXAMPLES

Unless indicated otherwise in these Examples, the methods involving commercial kits were done following the instructions of the manufacturers.

### Example 1

S. aureus Detection: Growth and Phage Assay Conditions

Staphylococcus aureus (S. aureus) bacterial cultures were grown for one hour in LB-Lennox (10 g/l Tripone, 5 g/l yeast extract and 5 g/l NaCl) media at 37° C. with strong aeration (225 rpm shaking). For antibiotic sensitivity assays the cultures were grown for one or three hours, as indicated, and cefoxitin (Sigma-Aldrich) was added at a final concentration of 4 μg/ml using a 1,000× stock in water.

The reactions were performed by mixing 50 μl of S. aureus bacterial cultures (˜2×10⁹ cfu/ml) and 50 μl of a phage K lysate (about 5×10⁸ pfu/ml) or 1 μl of CsCl gradient purified phage K (about 1×10¹⁰ pfu/ml). The mixtures were incubated on ice for 3-4 minutes and the infections were allowed to proceed for 10 minutes, unless indicated otherwise, at 37° C. with shaking (400 rpm). Five minutes prior to the end of the incubation, lysostaphin (Sigma-Aldrich) was added from a 20× stock (2 mg/ml).

### Example 2

S. aureus Detection: Phage Lysates and CsCl Gradient Purification

Phage K lysates were done by infecting S. aureus K1 strain following standard procedures. The lysates were further processed by centrifugation at 3,500 g for 10 minutes and filtering the supernatant using 0.22 μm pore Steriflip filter units (Millipore). Phage purification using CsCl gradients were done as previously described (Estrella et al. (2016), “Characterization of novel Staphylococcus aureus lytic phage and defining their combinatorial virulence using the OmniLog system,” Bacteriophage, 6:e1219440).

### Example 3

S. aureus Detection: RNA Preps, DNA Preps, Gel Electrophoresis and Sequencing

RNA preps were done using the hot-phenol method (Köhrer et al. (1991), “Preparation of high molecular weight RNA,” Methods Enzymol., 194:398-405) followed by treatment with amplification grade DNAse I (Thermo Fisher Scientific), or using Direct-zol RNA MicroPrep with TRI Reagent kits (Zymoresearch). Phage DNA preps were done by proteinase K treatment followed by phenol/chloroform extraction as indicated previously (Estrella et al. (2016), “Characterization of novel Staphylococcus aureus lytic phage and defining their combinatorial virulence using the OmniLog system,” Bacteriophage, 6:e1219440). Nucleic acid integrity was evaluated by gel electrophoresis in ethidium bromide agarose gels. 1 kb plus DNA ladder (Thermo Fisher Scientific) was used as a size marker. DNA band isolation was done using Purelink quick gel extraction kit (Thermo Fisher Scientific). Sanger sequencing was done by MacrogenUSA using the oligonucleotides OK1-F and SeqI2-F, and the resulting peaks were visualized using open source ApE plasmid editor software (Davis (2017) ApE, a plasmid editor. [retrieved in 2016] from the world wide web at biologylabs.utah.edu/jorgensen/wayned/ape].

### Example 4

S. aureus Detection: PCR and qPCR Reactions and Analysis

Regular polymerase chain reactions (PCRs) were done using Phusion High-Fidelity DNA polymerase (New England Biolabs) and a C1000 thermal cycler (BioRad). Stepwise reverse transcription (RT)-PCRs were done using SuperScript III First-Strand Synthesis System (Thermo Fisher Scientific) and the oligonucleotide OK1-R (SEQ ID NO: 2) for priming, followed by thermal amplification with Phusion high-fidelity PCR kit (NEB). The PCR conditions using the oligonucleotides OK1-F and OK1-R (SEQ ID NOS: 1 and 2) were: 98° C. 2 minutes/35× (98° C. 30 seconds, 50° C. 30 seconds, 72° C. 6 minutes)/72° C. 10 min. When using primer pairs OK2-F and OK2-R (SEQ ID NOS: 4 and 5) or OK3-F&-R (SEQ ID NOS: 6 and 7) the elongation time was reduced to 1 minute.

Single-step fluorescent quantitative or real-time PCR (qPCR) was done with Luna one-step RT-qPCR kit (NEB) using low profile clear 0.2 ml PCR tubes and optical ultraclear flat caps (BioRad). The reactions were run in a CFX96 real-time PCR detection system (BioRad) with CFX Manager 3. Quantification cycle values (Cqs) were auto calculated by the software, and ranged from about 190 to about 250. The single step RT-qPCR conditions are: 55° C. 10 minutes/35× (95° C. 10 seconds, 60° C. 30 seconds, plate read).

### Example 5

S. aureus Detection: Oligonucleotides

All the primers were designed manually based on the Genebank phage K sequence NC_005880 (Gill (2014), “Revised Genome Sequence of Staphylococcus aureus Bacteriophage K,” Genome Announc, 2:e01173-13 and O'Flaherty et al. (2004), “Genome of staphylococcal phage K: a new lineage of Myoviridae infecting gram-positive bacteria with a low G+C content” J Bacteriol., 186:2862-71), and synthetized by GeneOracle. The oligonucleotides used were: OK1-F (5′-GAGTTGGTAGATAACATTG-3′) (SEQ ID NO: 1), OK1-R (5′-AATTGTTCATCTGTTAGTTTACCTGACTCTTTATAATC-3′) (SEQ ID NO: 2), SeqI2-F (5′-CTACAATGAGAGAGCACTGG-3′) (SEQ ID NO: 3), OK2-F (5′-GAATTGCTTAAGAATTGGTTAGCTAG-3′) (SEQ ID NO: 4), OK2-R (5′-CCTGAATACTCTCAAAATCTTTAAAG-3′) (SEQ ID NO: 5), OK3-F (5′-ACAAGAAAGCTATTGGTTATGCGTTAGATAATITAG-3′) (SEQ ID NO: 6), and OK3-R (5′-TGTAGC-ATATCAGGGTCTTTAGTAAATAATCCGA-3′) (SEQ ID NO: 7).

### Example 6

S. aureus Detection: Bacterial Strains and Phage K

Phage K (Krueger et al. (1930), “The kinetics of the bacterium-bacteriophage reaction,” J Gen Physiol., 14:223-54) and the following bacterial strains were obtained from ATCC: S. aureus strain K1 (Raiston et al. (1954), “The isolation of a staphylococcal phage variant susceptible to an unusual host control,” J Gen Physiol., 37:685-716), Staphylococcus epidermidis Fussel strain (Hugh et al. (1968), “The neotype strain for Staphylococcus epidermidis (Winslow and Winslow 1908) Evans 1916,” Int J Syst Evol Microbiol., 18: 231-239, doi: 10.1099/00207713-18-3-231), Pseudomonas aeruginosa strain O1 (Holloway (1955), “Genetic recombination in Pseudomonas aeruginosa,” J Gen Microbiol., 13:572-581). The following bacteria are maintained in the inventors lab collection: S. aureus strain RN4220 strain (Kreiswirth et al. (1983), “The toxic shock syndrome exotoxin structural gene is not detectably transmitted by a prophage,” Nature, 305:709-12), Staphylococcus haemolyticus, SA1415.COI, SA1012.COI, SA1107.COI and SA1019.COI (Estrella et al. (2016), “Characterization of novel Staphylococcus aureus lytic phage and defining their combinatorial virulence using the OmniLog system,” Bacteriophage, 6:e1219440), and Acinetobacter baumannii strain AB5075 (Biswajit Biswas, Naval Medical Research Center, Fort Detrick, Frederick, Md.).

### Example 7

Detection of S. aureus by Phage K RT-PCR Plus Gel Electrophoresis

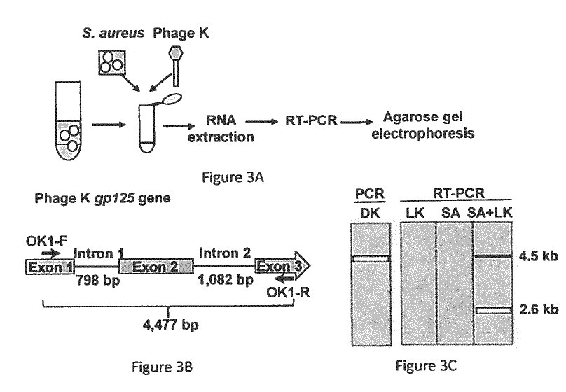

In order to detect S. aureus, the high metabolic rate of phages during infection was used advantageously. One exemplary embodiment of phage-mediated molecular detection (PMMD) of S. aureus included in the infection of the bacteria with phage K followed by detection of phage RNA, as illustrated in FIG. 3A. In overview, the S. aureus sample is incubated with phage K, pure RNA is then extracted, reverse transcribed and subjected to PCR amplification. S. aureus gp125 RNA was selected as the amplification target (FIG. 3B). This RNA encodes for a putative DNA polymerase and contains two group I introns (O'Flaherty et al. (2004), “Genome of staphylococcal phage K: a new lineage of Myoviridae infecting gram-positive bacteria with a low G+C content,” J Bacteriol., 186: 2862-71). The targeting of an intron-containing gene provided a QC point for the RT-PCR via analysis of the product size. The oligo OK1-R (SEQ ID NO: 2) was designed for reverse transcription (RT) priming, a step followed by PCR amplification with the oligos OK1-F and OK1-R (SEQ ID NOS: 1 and 2), as further described herein.

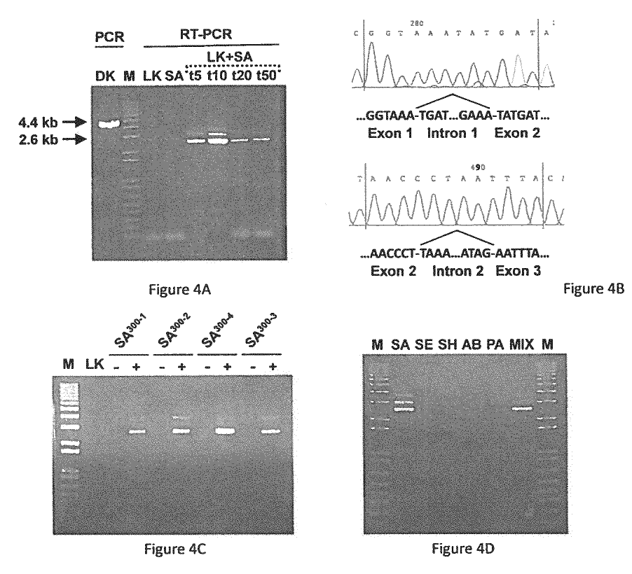

Under the conditions described above, it was expected that the amplification of a major 2.6 kb band, corresponding to the spliced RNA (mRNA), in the samples would indicate that infection had occurred as shown in FIG. 3C. As also shown in FIG. 3C, minor upper size band(s), corresponding to unspliced (pre-mRNA) and/or incorrectly spliced RNA also appear. In contrast, regular PCR of genomic phage K DNA should produce a single band of 4.6 kb. The empirical application of the assay with the SA RN4220 strain, as further described herein, fully agreed with this expectation (FIG. 4A), and revealed that an optimal incubation time is about 10 minutes in this exemplary embodiment. The partial sequencing of the 2.6 kb product with oligonucleotides specific for the area around the introns further confirmed that the diagnostic band results from the splicing of introns 1 and 2, and agreed with the predicted exon junctions at the nucleotide level (FIG. 4B) (O'Flaherty et al. (2004), “Genome of staphylococcal phage K: a new lineage of Myoviridae infecting gram-positive bacteria with a low G+C content,” J Bacteriol., 186: 2862-71).

### Example 8

S. aureus Detection: Assay Specificity

Typically, the PMMD methods described herein are dependent on the specificity of the phage. In the case of S. aureus, for example, the diagnostic assessment of clinically relevant isolates was used to evaluate embodiments of this method. To illustrate, a set of four clinical isolates belonging to the USA300 series, the most common community-associated methicillin-resistant group in the USA (Carrel et al. (2015), “USA300 methicillin-resistant Staphylococcus aureus, United States, 2000-2013,” Emerg Infect Dis 21:1973-1980. Doi: 10.3201/eid2111.150452), previously characterized as phage K sensitive (Estrella et al. (2016), “Characterization of novel Staphylococcus aureus lytic phage and defining their combinatorial virulence using the OmniLog system,” Bacteriophage, 6:e1219440) were assessed. As shown in FIG. 4C, all samples tested positive in this embodiment. To further illustrate, to evaluate an exemplary PMMD method using wound swabs, or other types of clinical specimen collection methods, the specificity and influence of the presence of additional bacteria in the culture were assessed. Therefore, these exemplary methods were tested with bacteria that are close relatives to S. aureus (Staphylococcus epidermidis (SE) and Staphylococcus haemolyticus (SH)) and with gram-human bacterial pathogens (Acinetobacter baumannii (AB) and Pseudomonas aeruginosa (PA)). In all cases tested, the exemplary method produced no false positive signals for non-S. aureus and detected S. aureus in complex mixtures (FIG. 4D).

### Example 9

### S. aureus Detection: Limit of Detection (LOD) Using Fluorescent Quantitative RT-PCR

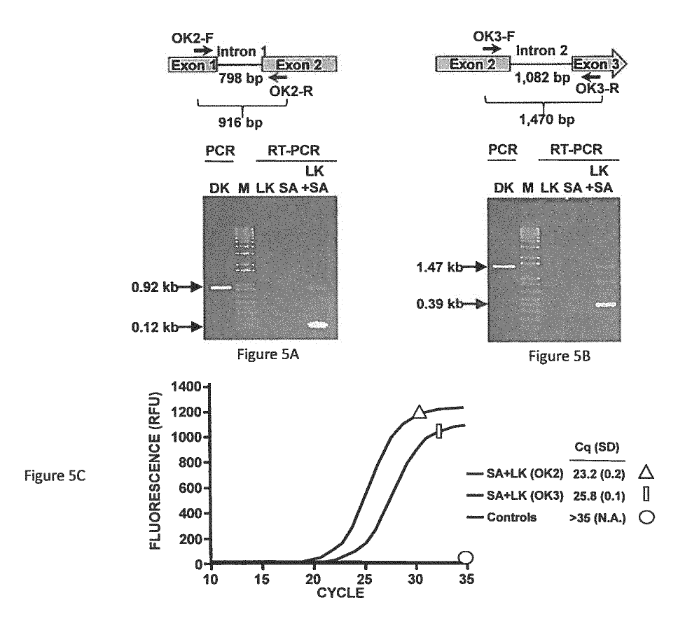

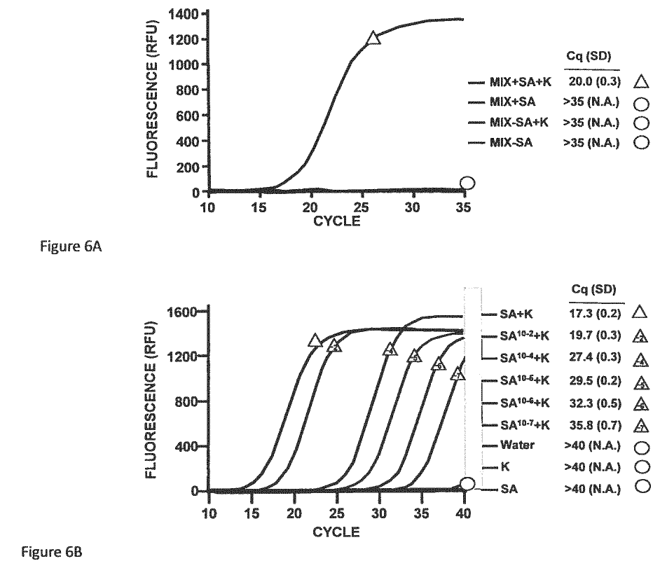

PCR analysis by gel electrophoresis is typically semi-quantitative and, although easy to perform and inexpensive, is generally not a method of choice for most clinical laboratories. To improve the workflow by analyzing the RT samples using fluorescent qPCR, the size of the diagnostic amplicon was decreased. The assay was repeated using oligonucleotides specific for intron 1 or intron 2 splicing events (FIGS. 5 A and B), obtaining in both cases the expected results. Analysis of single-step fluorescent qPCR of the same amplicons (FIG. 5C) indicated that both primer pairs effectively produced diagnostic signals, and that the signals specific for intron 1 splicing is stronger. The primer pairs described herein were used in these exemplary embodiments. The specificity results were confirmed using mixed cultures of different uninfected bacteria, as illustrated in FIG. 4D. They did not produce false positives. On the other hand, mixed cultures (MIX) incubated with phage K (+K) resulted in a prominent signal only when S. aureus is present in the culture (FIG. 6A). This validates the RT-qPCR approach for detection of S. aureus in complex samples.

An important factor for the successful clinical application of certain PMMD embodiments when inoculating directly using clinical sampling devices, e.g. a swab from a wound, is the ability to detect a relatively small number of bacteria. With the process described herein, involving hot-phenol acid RNA extraction and the use of an unpurified lysate of phage K, a reliable strong signal was obtained with about 10,000 bacteria (not shown). The LOD was increased by using a column-based RNA purification method (Direct Zol RNA prep Kit, as described further herein) (FIG. 6B). The use of this kit not only improves the LOD but also simplifies and speeds up the RNA extraction step. It is noteworthy that in order to increase the number of cycles from 35 to 40 without compromising the assay with phage-only signal, CsCl gradient purified phage K was used instead of the phage lysates. Under these conditions and applying the exemplary PMMD method to cultures of S. aureus diluted at different degrees, the assay sensitivity is about 100 cfu (FIG. 6B), a number of bacteria that is significantly below the expected presence in clinical sampling of infected tissues.

### Example 10

### S. aureus Detection: Discrimination of MSSA Versus MRSA

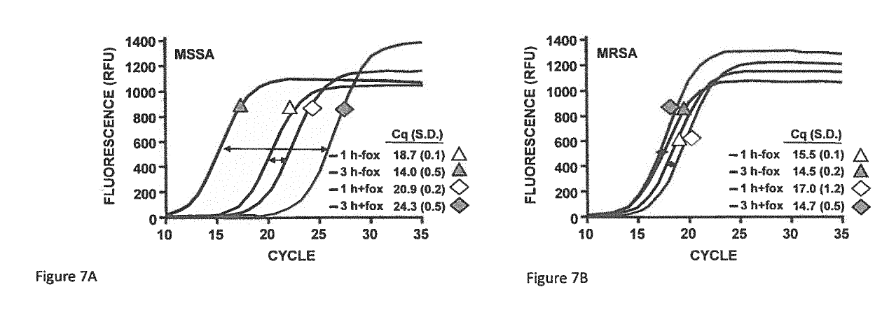

Small adjustments of the exemplary PMMD method allow this technique to be used for applications other than detection alone. Since the initial step used an incubation time of about 1 hour for the bacteria to increase its metabolic rate, it seemed that MSSA and MRSA could be differentiated from one another if the cultures were incubated with and without cefoxitin (fox), an antibiotic used for susceptibility testing of MRSA isolates (Palavecino (2014), “Clinical, epidemiologic, and laboratory aspects of methicillin-resistant Staphylococcus aureus infections,” Methods Mol Biol., 1085:1-24 and Swenson et al. (2005) “Results of disk diffusion testing with cefoxitin correlate with presence of mecA in Staphylococcus spp,” J Clin Microbiol., 43:3818-23). As shown in FIG. 7A, the addition of fox in the media increased the Cq values in the MSSA strain RN4220 by about 1.5 and about 11.5 after one and three hours of incubation respectively. By contrast, for the MRSA strain SA-1012.COI, the presence of fox only resulted in Cq increases of about 1.0 and about 0.0 under the same conditions. The period of time for the proper induction of the antibiotic resistant gene(s) is the most likely explanation for the effect of fox in SA-1012.COI after 1 hour incubation. Therefore, the comparison of PMMD results after incubation with and without fox prior to infection can be used for determination of methicillin resistance. Moreover, embodiments of the PMMD method are readily adaptable for assessing other drugs, either bactericide or bacteriostatic, since, for example, the active growth of the bacteria is typically needed for phage metabolism (Krueger et al. (1930), “The kinetics of the bacterium-bacteriophage reaction,” J Gen Physiol., 14:223-54).

### Example 11

### S. aureus Detection: Phage-Mediated Molecular Detection Bacteriophage K “Resistant” Strains

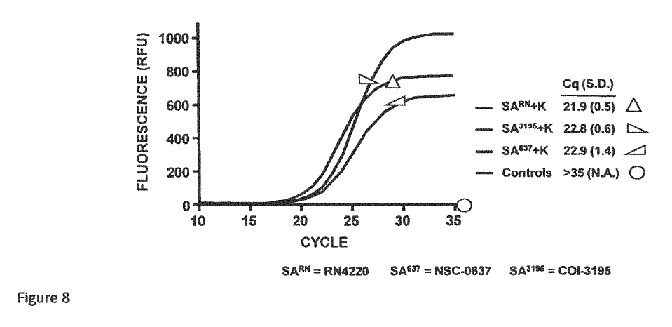

The lab strain RN4220 along with two strains, COI-3195 and NSC-0637, resistant to phage K using plaque assays (Estrella et al. (2016), “Characterization of novel Staphylococcus aureus lytic phage and defining their combinatorial virulence using the OmniLog system,” Bacteriophage, 6:e1219440; and retested in this example) were subjected to PMMD as in the other examples involving the detection of S. aureus described herein. As shown in FIG. 8, PMMD produced very strong positive signals even with strains that are classified as resistant by plaque assays. The PMMD method advantageously uses the efficient initial steps of the first infective phage life cycle. In contrast, the plaque assays require that the whole life cycle not be compromised and that a number of infection-bursts generations occur. Therefore, the PMMD detection spectrum for S. aureus strains is even greater that previously determined for phage K using other assays.

### Example 12

### E. coli and B. anthracis Detection: Additional Exemplary Applications of PMMD Methods

To further show the ready adaptability of the PMMD methods for the detection and antibiotic sensitivity assessment of other bacterial organisms beyond S. aureus, a similar approach as described herein was applied for Escherichia coli (E. coli) and Bacillus anthracis (B. anthracis) using the bacteriophages T4 and Gamma, respectively. For E. coli, primer nucleic acids specific for the nrdB gene of T4 phage were used. The specific sequences are as follows: T4nrdB-f (5′-CTTTACCTCTGTTTGCACGTT-3′) (SEQ ID NO: 8) and T4nrdB-rev (5′-CATTACCTTCCATGATTTCCATGT-3′) (SEQ ID NO: 9). Similar to the examples involving the detection of S. aureus described herein, the oligonucleotides amplified a spliced RNA (T4 nrdB RNA) to produce a fragment of 109 bp in length if spliced (707 bp if unspliced). In addition, the E. coli strain used in this example was W3110 (a K12 strain): F lambda IN(rrnD-rrnE)1 rph-1 (Genome NC_007779).

For B. anthracis, primer nucleic acids that amplify a 118 bp fragment of the dnaC Gamma phage were used. The specific sequences are as follows: Gamma pol-f (5′-GTATACATTGCAGGGATTCAAG-3′) (SEQ ID NO: 10) and Gamma pol-rev (5′-CATGTTCGTCATTTAATTTCTCACC-3′) (SEQ ID NO: 11). The B. anthracis strain used for this example was the “Sterne” strain (strain 7702): [retrieved in 2016], which is described on the world wide web at ncbi.nlm.nih.gov/bioproject/PRJNA10878. See also, Rasko D A et al., “Genomics of the Bacillus cereus group of organisms,” FEMS Microbiol Rev, 2005 April; 29(2):303-29. Since RNA is typically more difficult to extract from B. anthracis than for other bacterial species, such as E. coli, the protocol was slightly different than the extraction procedure used for S. aureus and E. coli.

In the case of T4 phage infection of E. coli, rather than using lysostaphin such as described with regard to phage K infection S. aureus, lysozyme was used. Specifically, five minutes prior to the end of the incubation of T4 and E. coli, lysozyme (Sigma-Aldrich) was added from a 10× stock (100 mg/ml).

In the case of Gamma phage infection of B. anthracis, the incubation of phage and bacteria was not subject to enzymatic treatment. Instead, after the 10 minutes incubation, 25 microliters of a “lysis mix” (a mixture of 200 microliters of 10% SDS and 50 microliters of 0.5 M EDTA) was added to the samples, followed by 5-minute incubation at 99° C. This was followed by the addition of 100 microliters of acid phenol and incubation at 65° C. for 1 hour. We note herein that trizol treatment and TRI Reagent kits (Zymoresearch) were not employed due to inefficient efficacy for B. anthracis (tested empirically in our lab). Aside from this step, RNA extraction proceeded as described herein for infected S. aureus samples.

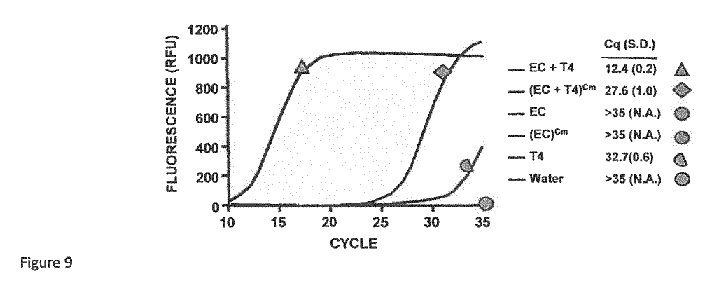

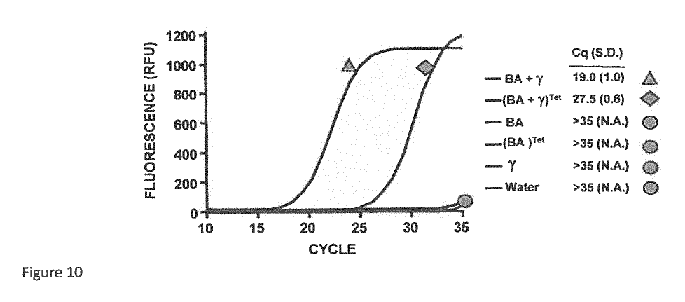

In addition to bacterial detection, antibiotic sensitivity was also evaluated. For E. coli, chloramphenicol (Cm) was used, while for B. anthracis, tetracycline (Tet) was used. Otherwise, the molecular protocol used was similar to the one utilized for S. aureus detection and antibiotic sensitivity assessment as described herein. The results of these assays are shown in FIG. 9 for E. coli and in FIG. 10 for B. anthracis, which illustrate the robustness of the PMMD methods in these applications. In both cases, no signal was observed without phage interaction.

All patents, patent applications, and published references cited herein are hereby incorporated by reference in their entirety. While this invention has been particularly shown and described with references to exemplary embodiments thereof, it will be understood by those skilled in the art that various changes in form and details may be made therein without departing from the scope of the invention encompassed by the appended claims.

1. A method of detecting an actively growing bacterial organism, the method comprising:
contacting a bacterial organism with an unmodified bacteriophage that infects the bacterial organism and produces one or more ribonucleic acid (RNA) products; and,
detecting at least one of the RNA products from the unmodified bacteriophage before the unmodified bacteriophage substantially replicates in the infected bacterial organism, thereby detecting the actively growing bacterial organism.

2. The method of claim 1, further comprising growing the bacterial organism in a culture medium prior to the contacting step.

3. The method of claim 1, wherein the duration of the contacting step is about 10 minutes or less prior to detecting the RNA products from the unmodified bacteriophage in the detecting step.

4. The method of claim 1, further comprising contacting one or more additional samples of the bacterial organism with a chosen antibiotic agent prior to detecting the RNA products, wherein detection of the RNA products from the unmodified bacteriophage in said samples at levels comparable to those detected in the infected bacterial organisms that have not been contacted with the chosen antibiotic agent indicates that the bacterial organism is resistant to the antibiotic agent.

5. The method of claim 4, wherein the one or more additional samples of the bacterial organism is contacted with the chosen antibiotic agent prior to contacting the one or more additional samples of the bacterial organism with the unmodified bacteriophage.

6. The method of claim 1, comprising lysing the bacterial organism after the contacting step and prior to the detecting step.

7. The method of claim 1, comprising purifying the RNA products produced by the unmodified bacteriophage prior to the detecting step.

8. The method of claim 1, comprising amplifying the RNA products produced by the unmodified bacteriophage prior to the detecting step.

9. The method of claim 1, wherein the bacterial organism is Staphylococcus aureus, the unmodified bacteriophage is Bacteriophage K, and the RNA products produced by the unmodified bacteriophage are amplified prior to or during the detecting step.

10. The method of claim 9, wherein the RNA products are amplified using one or more oligonucleotides comprising sequences selected from the group consisting of: SEQ ID NO: 1, SEQ ID NO: 2, SEQ ID NO: 3, SEQ ID NO: 4, SEQ ID NO: 5, SEQ ID NO: 6, and SEQ ID NO: 7.

11. The method of claim 1, wherein the bacterial organism is Escherichia coli, the unmodified bacteriophage is Enterobacteria phage T4, and the RNA products produced by the unmodified bacteriophage are amplified prior to or during the detecting step.

12. The method of claim 11, wherein the RNA products are amplified using one or more oligonucleotides comprising sequences selected from the group consisting of: SEQ ID NO: 8 and SEQ ID NO: 9.

13. The method of claim 1, wherein the bacterial organism is Bacillus anthracis, the unmodified bacteriophage is Bacillus anthracis Gamma phage, and the RNA products produced by the unmodified bacteriophage are amplified prior to or during the detecting step.

14. The method of claim 13, wherein the RNA products are amplified using one or more oligonucleotides comprising sequences selected from the group consisting of: SEQ ID NO: 10 and SEQ ID NO: 11.

15. A reaction mixture to detect Staphylococcus aureus, the reaction mixture comprising:
one or more ribonucleic acid (RNA) products from an unmodified Bacteriophage K, wherein the one or more RNA products have been obtained from Staphylococcus aureus that was infected with the unmodified Bacteriophage K; and,
one or more oligonucleotides comprising sequences selected from the group consisting of: SEQ ID NO: 1, SEQ ID NO: 2, SEQ ID NO: 3, SEQ ID NO: 4, SEQ ID NO: 5, SEQ ID NO: 6, and SEQ ID NO: 7.

16. A reaction mixture to detect Escherichia coli, the reaction mixture comprising:
one or more ribonucleic acid (RNA) products from an unmodified Enterobacteria phage T4, wherein the one or more RNA products have been obtained from Escherichia coli that was infected with the unmodified Enterobacteria phage T4; and,
one or more oligonucleotides comprising sequences selected from the group consisting of: SEQ ID NO: 8 and SEQ ID NO: 9.

17. A reaction mixture to detect Bacillus anthracis, the reaction mixture comprising:
one or more ribonucleic acid (RNA) products from an unmodified Bacillus anthracis Gamma phage, wherein the one or more RNA products have been obtained from Bacillus anthracis that was infected with the unmodified Bacillus anthracis Gamma phage; and,
one or more oligonucleotides comprising sequences selected from the group consisting of: SEQ ID NO: 10 and SEQ ID NO: 11.

18. A kit to detect an actively growing target bacterial organism, the kit comprising:
one or more containers comprising: (a) an unmodified bacteriophage that infects the target bacterial organism and produces one or more ribonucleic acid (RNA) products, and (b) one or more oligonucleotides to detect the RNA products; and,
instructions to contact the target bacterial organism with the unmodified bacteriophage to produce the RNA products and to detect the RNA products using the oligonucleotides.

19. The kit of claim 18, wherein the target bacterial organism is Staphylococcus aureus, the unmodified bacteriophage is Bacteriophage K, and the oligonucleotides comprise sequences selected from the group consisting of: SEQ ID NO: 1, SEQ ID NO: 2, SEQ ID NO: 3, SEQ ID NO: 4, SEQ ID NO: 5, SEQ ID NO: 6, and SEQ ID NO: 7.

20. The kit of claim 18, wherein the target bacterial organism is Escherichia coli, the unmodified bacteriophage is Enterobacteria phage T4, and the oligonucleotides comprise sequences selected from the group consisting of: SEQ ID NO: 8 and SEQ ID NO: 9.

21. The kit of claim 18, wherein the target bacterial organism is Bacillus anthracis, the unmodified bacteriophage is Bacillus anthracis Gamma phage, and the oligonucleotides comprise sequences selected from the group consisting of: SEQ ID NO: 10 and SEQ ID NO: 11.

22. The kit of any one of claims 18-21, further comprising instructions to contact one or more additional samples of the target bacterial organism with a chosen antibiotic agent prior to detecting the RNA products, wherein detection of the RNA products from the unmodified bacteriophage in said samples at levels comparable to those detected in the infected bacterial organisms that have not been contacted with the chosen antibiotic agent indicates that the target bacterial organism is resistant to the antibiotic agent.

23. The kit of claim 22, comprising at least one container comprising the chosen antibiotic agent.

24. A system to detect an actively growing bacterial organism, the system comprising:
a reaction mixture processing component configured to contact a bacterial organism with an unmodified bacteriophage that infects the bacterial organism and produces one or more ribonucleic acid (RNA) products; and,
a detection component configured to detect the RNA products from the unmodified bacteriophage before the unmodified bacteriophage substantially replicates in the bacterial organism to thereby detect the actively growing bacterial organism.

25. The system of claim 24, further comprising a reagent handling component configured to transfer one or more reagents to and/or from the reaction mixture processing component.

26. The system of claim 24, further comprising a sample preparation component configured to prepare one or more samples comprising the bacterial organism prior to being contacted with the unmodified bacteriophage and/or to purify the RNA products produced by the unmodified bacteriophage.

27. The system of claim 24, further comprising a controller comprising logic instructions that direct the reaction mixture processing component to contact the bacterial organism with the unmodified bacteriophage, and the detection component to detect the RNA products.

28. The system of claim 24, wherein the bacterial organism is Staphylococcus aureus, the unmodified bacteriophage is Bacteriophage K, and the detection component comprises a nucleic acid amplification component that is configured to amplify the RNA products using one or more primer oligonucleotides.

29. The system of claim 28, wherein the primer oligonucleotides comprise sequences selected from the group consisting of: SEQ ID NO: 1, SEQ ID NO: 2, SEQ ID NO: 3, SEQ ID NO: 4, SEQ ID NO: 5, SEQ ID NO: 6, and SEQ ID NO: 7.

30. The system of claim 24, wherein the bacterial organism is Escherichia coli, the unmodified bacteriophage is Enterobacteria phage T4, and the detection component comprises a nucleic acid amplification component that is configured to amplify the RNA products using one or more primer oligonucleotides.

31. The system of claim 30, wherein the primer oligonucleotides comprise sequences selected from the group consisting of: SEQ ID NO: 8 and SEQ ID NO: 9.

32. The system of claim 24, wherein the bacterial organism is Bacillus anthracis, the unmodified bacteriophage is Bacillus anthracis Gamma phage, and the detection component comprises a nucleic acid amplification component that is configured to amplify the RNA products using one or more primer oligonucleotides.

33. The system of claim 32, wherein the primer oligonucleotides comprise sequences selected from the group consisting of: SEQ ID NO: 10 and SEQ ID NO: 11.

34. A microfluidic card to detect actively growing bacterial organisms, the microfluidic card comprising:
a loading port configured to receive one or more samples comprising a bacterial organism;
a reaction chamber operably connected to the loading port, wherein said reaction chamber is configured to receive: (a) said one or more samples comprising the bacterial organism from the loading port and wherein the reaction chamber comprises, or is configured to receive, an unmodified bacteriophage, or (b) said one or more samples, wherein the sample further comprises an unmodified bacteriophage, such that the unmodified bacteriophage infects the bacterial organism and produces one or more ribonucleic acid (RNA) products when the reaction chamber receives the sample;
a detection area in which detectable signals indicative of the RNA products are produced when the detection area comprises the RNA products, wherein said detection area operably communicates with the reaction chamber; and,
a conveyance mechanism operably connected at least to the reaction chamber, wherein said conveyance mechanism is configured to convey the sample and/or other reagents to and/or from the reaction chamber.

35. The microfluidic card of claim 34, wherein the reaction chamber comprises the detection area.

36. The microfluidic card of claim 34, wherein the bacterial organism is Staphylococcus aureus, the unmodified bacteriophage is Bacteriophage K, and the detection area comprises a nucleic acid amplification component that is configured to amplify the RNA products.

37. The microfluidic card of claim 36, wherein the nucleic acid amplification component is configured to amplify the RNA products using one or more oligonucleotides comprising sequences selected from the group consisting of: SEQ ID NO: 1, SEQ ID NO: 2, SEQ ID NO: 3, SEQ ID NO: 4, SEQ ID NO: 5, SEQ ID NO: 6, and SEQ ID NO: 7, when the nucleic acid amplification component comprises the oligonucleotides.

38. The microfluidic card of claim 34, wherein the bacterial organism is Escherichia coli, the unmodified bacteriophage is Enterobacteria phage T4, and the detection area comprises a nucleic acid amplification component that is configured to amplify the RNA products.

39. The microfluidic card of claim 38, wherein the nucleic acid amplification component is configured to amplify the RNA products using one or more oligonucleotides comprising sequences selected from the group consisting of: SEQ ID NO: 8 and SEQ ID NO: 9, when the nucleic acid amplification component comprises the oligonucleotides.

40. The microfluidic card of claim 34, wherein the bacterial organism is Bacillus anthracis, the unmodified bacteriophage is Bacillus anthracis Gamma phage, and the detection area comprises a nucleic acid amplification component that is configured to amplify the RNA products.

41. The microfluidic card of claim 40, wherein the nucleic acid amplification component is configured to amplify the RNA products using one or more oligonucleotides comprising sequences selected from the group consisting of: SEQ ID NO: 10 and SEQ ID NO: 11, when the nucleic acid amplification component comprises the oligonucleotides.


SEQUENCE LISTING (11 sequences)

SEQ ID NO: 1 - DNA, 19 bp, Artificial Sequence
  Primer Oligonucleotide for Staphylococcus aureus
gagttggtagataacattg

SEQ ID NO: 2 - DNA, 38 bp, Artificial Sequence
  Primer Oligonucleotide for Staphylococcus aureus
aattgttcatctgttagtttacctgactctttataatc

SEQ ID NO: 3 - DNA, 20 bp, Artificial Sequence
  Primer Oligonucleotide for Staphylococcus aureus
ctacaatgagagagcactgg

SEQ ID NO: 4 - DNA, 26 bp, Artificial Sequence
  Primer Oligonucleotide for Staphylococcus aureus
gaattgcttaagaattggttagctag

SEQ ID NO: 5 - DNA, 26 bp, Artificial Sequence
  Primer Oligonucleotide for Staphylococcus aureus
cctgaatactctcaaaatctttaaag

SEQ ID NO: 6 - DNA, 36 bp, Artificial Sequence
  Primer Oligonucleotide for Staphylococcus aureus
acaagaaagctattggttatgcgttagataatttag

SEQ ID NO: 7 - DNA, 34 bp, Artificial Sequence
  Primer Oligonucleotide for Staphylococcus aureus
tgtagcatatcagggtctttagtaaataatccga

SEQ ID NO: 8 - DNA, 21 bp, Artificial Sequence
  Primer Oligonucleotide for Escherichia coli
ctttacctctgtttgcacgtt

SEQ ID NO: 9 - DNA, 24 bp, Artificial Sequence
  Primer Oligonucleotide for Escherichia coli
cattaccttccatgatttccatgt

SEQ ID NO: 10 - DNA, 22 bp, Artificial Sequence
  Primer Oligonucleotides for Bacillus anthracis
gtatacattgcagggattcaag

SEQ ID NO: 11 - DNA, 25 bp, Artificial Sequence
  Primer Oligonucleotide for Bacillus anthracis
catgttcgtcatttaatttctcacc